(19) United States
(12) Patent Application Publication
(10) Pub. No.: US 2026/0232573 A1
(43) Pub. Date: Aug. 13, 2026

(54) DEGARELIX ORGANIC SOLVENT FORMULATIONS

(71) Applicant: TOLMAR INTERNATIONAL LIMITED, Dublin 2 (IE)

(72) Inventors: John Downing, Fort Collins, CO (US); Bennet Harding, Fort Collins, CO (US); Dileep Janagam, San Ramon, CA (US); Jinqiu Xu, Fort Collins, CO (US); David Loffredo, Elmhurst, IL (US); Natalie Akagi Bath, Fort Collins, CO (US)

(21) Appl. No.: 19/154,569

(22) PCT Filed: Feb. 7, 2024
(86) PCT No.: PCT/US2024/014782

§ 371 Date: Aug. 7, 2025

Related U.S. Application Data
(60) Provisional application No. 63/484,436, filed on Feb. 10, 2023.
(60) Provisional application No. 63/613,236, filed on Dec. 21, 2023.

(51) Int. Cl. A61K 9/00; A61K 38/09; A61K 47/10; A61K 47/12

(52) U.S. Cl. CPC A61K 9/0024; A61K 38/09; A61K 47/10; A61K 47/12

(57) ABSTRACT

Described herein are long-acting injectable compositions as well as methods of using such long-acting injectable compositions. The long-acting injectable compositions comprise degarelix (a decapeptide gonadotropin-releasing hormone (GnRH) receptor antagonist) or a pharmaceutically acceptable salt thereof. These compositions can also comprise a biocompatible organic solvent, optionally with one or more additives in a single syringe system.

### CROSS-REFERENCE TO RELATED APPLICATION

This application claims the benefit of priority of U.S. Provisional Patent Application 63/484,436, filed 10 Feb. 2023 and U.S. Provisional Patent Application 63/613,236 filed 21 Dec. 2023, the entirety of each of which is incorporated herein by reference.

### FIELD

This invention relates generally to long-acting injectable degarelix/organic solvent formulations.

### BACKGROUND

Degarelix is a decapeptide gonadotropin-releasing hormone (GnRH) receptor antagonist indicated for the treatment of patients with advanced prostate cancer. Degarelix acts by directly blocking GnRH action on the pituitary by competitively and reversibly binding to GnRH receptors and produces a fast suppression of testosterone with no initial surge or minimal surge.

The currently marketed drug-delivery technology for degarelix in the United States and Europe (e.g., FIRMAGON® (degarelix for injection), Ferring Pharmaceuticals Inc. or Ferring GmbH) has significant limitations, including administration of a starting dose given as two high volume injections (3 mL each), followed by maintenance doses administered as a single high volume injection (1×4 mL) every 28 days. The only commercially available formulation with a dosing interval greater than once monthly is marketed only in Japan (GONAX® (degarelix for injection), Astellas Pharma Inc.), and has similar significant limitations, including administration of a starting dose given as two injections of 3 mL each, followed by a maintenance dose given as two injections of 4 mL each every 12-weeks. Thus, for all currently available degarelix products, multiple, high-volume injections (a total of 4-8 mLs per dosing period) are needed for extended therapy, which are associated with injection-related pain. Furthermore, degarelix has a tendency to self-aggregate in aqueous media. In the case of FIRMAGON®, degarelix is supplied as a powder (lyophilized, contained mannitol as a bulking agent), and once it is reconstituted with water the degarelix molecules aggregate and cross-link in a gel-forming network, resulting in a hydrogel. Because of its self-aggregation property and chemical instability in aqueous media, it has to be separated out from the aqueous vehicle and needs to be kept in a dried form by lyophilization. Therefore, it needs to be reconstituted at the time of administration. Since the rate and extent of self-aggregation/gel formation of peptide in aqueous media is concentration dependent, it is challenging to prepare a ready-to-inject formulation of degarelix for injection. In addition, the commercially available form of degarelix is manufactured by aseptic processing and involves multiple steps for reconstitution.

There is an unmet need for a long-acting GnRH antagonist formulation that is easy to administer, can be terminally sterilized during manufacturing, is administered less frequently than once per month, reduces the overall number of injections in a single dosing period, significantly reduces the injection volume, and reduces pain upon injection. Thus, it would be highly beneficial to have an injectable drug formulation of degarelix in a single syringe system that is ready-to-inject, and provides in a single injection, extended-release of a therapeutically effective amount of degarelix for more than a month within a low injection volume (<2.5 mL or ≤2 mL).

### SUMMARY

In various embodiments, a composition comprising a therapeutically effective amount of degarelix or a pharmaceutically acceptable salt thereof is disclosed. The composition comprises an organic biocompatible solvent, wherein the composition is formulated for subcutaneous injection into a subject; wherein a single dose of the composition is about 2.5 mL or less, or about 2.0 mL or less; and upon injection into the subject, the composition forms an in situ depot that releases the degarelix over a time period of from about 1 month to about 6 months.

In one aspect of the composition, the in situ depot releases the degarelix over a time period selected from the group consisting of: at least about 1 month, at least about 1.5 months, at least about 2 months, at least about 2.5 months, at least about 3 months, at least about 3.5 months, at least about 4 months, at least about 4.5 months, and at least about 5 months.

In one aspect of the composition, the pharmaceutically acceptable salt of the degarelix is selected from degarelix acetate, degarelix citrate, degarelix pamoate, degarelix palmitate, and degarelix mesylate.

In one aspect of the composition, the amount of biocompatible solvent in the composition is from about 50% to about 99% by weight of the composition.

In one aspect of the composition, the therapeutically effective amount of degarelix or pharmaceutically acceptable salt in the composition is from about 1% to about 50% by weight of the composition.

In one aspect of the composition, the therapeutically effective amount of degarelix is from about 40 mg-500 mg.

In still another aspect of the composition, the therapeutically effective amount of degarelix is from about 80 mg-500 mg.

In yet another aspect of the composition, the therapeutically effective amount of degarelix is from about 120 mg-500 mg.

In one aspect of the composition, the biocompatible solvent is selected from the group consisting of: N-methyl-2-pyrrolidone (NMP), dimethyl sulfoxide (DMSO), dimethylacetamide (DMA), benzyl benzoate (BnBzO), polyethylene glycol 15 hydroxystearate, methyl ethyl ketone, methyl lactate, benzyl alcohol, propylene carbonate (PC), triacetin, tributyl citrate, acetyl tributyl citrate, acetyl triethyl citrate, triethyl citrate, diethylene glycol monomethyl ether, ethyl acetate, N-ethyl-2-pyrrolidone, glycofurol and combinations thereof. In one preferred aspect the biocompatible organic solvent is NMP or DMSO.

In one aspect of the composition, a single dose of the composition is about 2.5 mL or less, about 2.4 mL or less, about 2.3 mL or less, about 2.2 mL or less, about 2.1 mL or less, about 2.0 mL or less, about 1.9 mL or less, about 1.8 mL or less, about 1.7 mL or less, about 1.6 mL or less, about 1.5 mL or less, about 1.4 mL or less, about 1.3 mL or less, about 1.2 mL or less, about 1.1 mL or less, about 1 mL or less, about 0.75 mL or less, about 0.5 mL or less, or about 0.375 mL or less.

In one aspect of the composition, a single dose of the composition is about 1.0 mL or less.

In one aspect of the composition, the degarelix is dissolved or dispersed in the biocompatible solvent.

In one aspect of the composition, the composition has been terminally sterilized or sterile filtered. In one aspect, wherein the composition has been terminally sterilized by e-beam.

In one aspect of the composition, the composition further comprises one or more additives. In one aspect, the additive is selected from the group consisting of polysorbate 20, polysorbate 80, poloxamer 188, sorbitan trioleate, lecithin (e.g., soya or egg), polyethylene glycol (PEG), PEG 300, 2-pyrrolidone, alpha-tocopherol, Vitamin E TPGS, sucrose cocoate, sucrose stearate, sucrose laurate, proline, arginine, sodium metabisulfite butylated hydroxyanisole, butylated hydroxyquinone, butylhydroxyanisol, hydroxycoumarin, butylated hydroxytoluene, cephalm, ethyl gallate, propyl gallate, octyl gallate, lauryl gallate, propyl-hydroxybenzoate, trihydroxybutylrophenone, vitamin E, lecithin, ethanolamine, ZnCl₂, MgCl₂, CaCl₂), DL-methioninecitric acid, dimethylphenol, dibutylphenol, ethylenediaminotetraacetic acid (EDTA), ethylene glycol-bis(β-aminoethyl ether)-N,N,N′,N′-tetraacetic acid (EGTA), ascorbic acid, nitrilotriacetic acid, n-hydroxyethylethylenediaminetriacetic acid (HEDTA), mercaptoethanol, and combinations thereof.

In another aspect, the additive is an acid additive. In one aspect, the acid additive is selected from the group consisting of acetic acid (AcOH), citric acid, succinic acid, methane sulfonic acid, sulfuric acid, hydrochloric acid (HCL), pamoic acid, palmitic acid, hydrobromic acid, nitric acid, chromic acid, trifluoroethanes sulfonic acid, trichloroacetic acid, dichloroacetic acid, bromoacetic acid, chloroacetic acid, cyanoacetic acid, 2-chloropropanoic acid, 4-cyanobutanoic acid, perchloric acid, phosphoric acid, hydrogen iodide, and combinations thereof. In another aspect, the acid additive is selected from the group consisting of acetic acid, citric acid and succinic acid.

In still another aspect, the additive is an alcohol additive. In one aspect, the alcohol additive is benzyl alcohol (BnOH).

In one aspect of the composition, the amount of the acid additive in the composition is from about 0.1 wt % to about 10.0 wt %.

In one aspect of the composition, the amount of the alcohol additive in the composition is from about 1.0 wt % to 30 wt %.

In one embodiment, a pharmaceutical composition, comprising about 20 wt % to 40 wt % of degarelix acetate or degarelix citrate; and about 60 wt % to 80 wt % of N-methyl-2-pyrrolidone (NMP) or dimethyl sulfoxide (DMSO) is disclosed. In one aspect of this embodiment, the composition further comprises about 0.1 wt % to 10 wt % of an acid additive. In one aspect, the acid additive is selected from the group consisting of acetic acid, citric acid, and succinic acid.

In one embodiment, a pharmaceutical composition, comprising about 25 wt % to 45 wt % of degarelix acetate or degarelix citrate; and about 55 wt % to 75 wt % of N-methyl-2-pyrrolidone (NMP) or dimethyl sulfoxide (DMSO) is disclosed. In one aspect of this embodiment, the composition further comprises about 1.0 wt % to 30 wt % of an alcohol additive. In one aspect, the alcohol additive is benzyl alcohol (BnOH).

In one embodiment, a pharmaceutical composition, comprising about 35 wt % of degarelix acetate; and about 65 wt % of N-methyl-2-pyrrolidone (NMP) is disclosed.

In one embodiment, a pharmaceutical composition, comprising about 35 wt % of degarelix acetate and about 65 wt % of DMSO is disclosed.

In one embodiment, a pharmaceutical composition, comprising about 35 wt % of degarelix acetate; about 60 wt % of NMP; and about 5 wt % of benzyl alcohol (BnOH) is disclosed.

In one embodiment, a pharmaceutical composition, comprising about 35 wt % of degarelix citrate; and about 65 wt % of NMP is disclosed.

In one embodiment, a pharmaceutical composition, comprising about 35 wt % of degarelix acetate; about 60 wt % to about 64 wt % of NMP; and about 1 wt % to about 5 wt % AcOH is disclosed. In one aspect, the NMP is in an about of about 62 wt % to about 64 wt %, and the amount of AcOH is about 1 wt % to about 3 wt %. In still another aspect, the NMP is in an amount of about 62.8 wt %, and the amount of AcOH is about 2.2 wt %.

In any of the above embodiments, in one aspect, the amount of degarelix or the pharmaceutically acceptable salt thereof in the pharmaceutical composition is from about 30 wt % to about 35 wt % degarelix free base equivalent. In one aspect, the amount of degarelix or the pharmaceutically acceptable salt thereof in the pharmaceutical composition is from about 31 wt % to about 34 wt % degarelix free base equivalent. In one aspect, the amount of degarelix or the pharmaceutically acceptable salt thereof in the pharmaceutical composition is from about 31 wt % to about 33 wt % degarelix free base equivalent. In one aspect, the amount of degarelix or the pharmaceutically acceptable salt thereof in the pharmaceutical composition is from about 31 wt % to about 32 wt % degarelix free base equivalent. In some aspects, when a target dose to be administered to a subject is calculated based on degarelix free base equivalent in the composition, the percent by weight of solvent, and the percent by weight of additive if included, may be modified accordingly.

In one embodiment, a pharmaceutical composition, comprising about 24 wt % of degarelix acetate; about 56 wt % of NMP; and about 20 wt % of BnOH is disclosed.

In one embodiment, a pharmaceutic composition comprising about 24 wt % of degarelix acetate; about 66 to 75 wt % NMP; and about 1 to 10 wt % AcOH is disclosed.

In one embodiment, a pharmaceutic composition comprising about 24 wt % of degarelix acetate; about 70 to 75 wt % NMP; and about 1 to 6 wt % AcOH is disclosed.

In one embodiment, a pharmaceutical composition, comprising about 24 wt % of degarelix acetate; about 73.3 wt % of NMP; and about 1.7 wt % of AcOH is disclosed.

In any of the above embodiments, in one aspect, the amount of degarelix or the pharmaceutically acceptable salt thereof in the pharmaceutical composition is from about 20 wt % to about 23 wt % degarelix free base equivalent. In one aspect, the amount of degarelix or the pharmaceutically acceptable salt thereof in the pharmaceutical composition is from about 20 wt % to about 22 wt % degarelix free base equivalent. In one aspect, the amount of degarelix or the pharmaceutically acceptable salt thereof in the pharmaceutical composition is from about 21 wt % to about 23 wt % degarelix free base equivalent. In one aspect, the amount of degarelix or the pharmaceutically acceptable salt thereof in the pharmaceutical composition is from about 21 wt % to about 22 wt % degarelix free base equivalent. In some aspects, when a target dose to be administered to a subject is calculated based on degarelix free base equivalent in the composition, the percent by weight of solvent, and the percent by weight of additive if included, may be modified accordingly.

In various embodiments, methods of treating prostate cancer and central precocious puberty (CPP) as well as methods of reducing serum testosterone levels and suppressing ovarian function by subcutaneously administering the compositions disclosed herein to subjects in need thereof is also contemplated.

In one embodiment, a method of treating prostate cancer in a subject, comprising subcutaneously administering the compositions disclosed herein to the subject is disclosed. In one aspect, the prostate cancer is advanced prostate cancer. In one aspect, the dose amount of degarelix or pharmaceutically acceptable salt in the composition is administered in a dose amount of about 40 mg to about 500 mg.

In one embodiment, a method of reducing serum testosterone levels in a subject to 50 ng/dL or less, comprising subcutaneously administering the compositions disclosed herein to the subject is disclosed. In one aspect, the serum testosterone level is below 20 ng/dL. In still another aspect, the serum testosterone level is below 10 ng/dL.

In one embodiment, a method of suppressing ovarian function in a subject with hormone-receptor positive breast cancer, comprising subcutaneously administering the compositions disclosed herein to the subject is disclosed. In one aspect, the hormone receptor positive breast cancer is estrogen receptor (ER) positive breast cancer. In one aspect, the subject's estradiol (E2) production level is suppressed to a level less than about 20 pg/mL to about less than about 2 pg/mL. In one aspect, the subject's follicle stimulating hormone (FSH) level is suppressed to a level less than about 40 IU/L. In one aspect, the subject's luteinizing hormone (LH) level is suppressed to a level less than about 4 IU/L.

In one embodiment, a method of treating central precocious puberty (CPP) in a subject comprising subcutaneously administering the compositions disclosed herein to the subject is disclosed. In one aspect, the subject's CPP blood serum LH concentration level is reduced to a pre-pubertal concentration level of less than about 4 IU/L.

In any aspects of the methods disclosed herein, the composition is administered about once every 1 month, about once every 2 months, about once every 3 months, about once every 4 months, about once every 5 months, or about once every six months.

In any aspects of the methods disclosed herein, the composition is administered to the subject about once every three months.

In any aspects of the methods disclosed herein, the composition is administered as a loading dose followed by a maintenance dose of the composition about 1 month, 2 months or 3 months after the loading dose has been administered. In one aspect, when a loading dose is administered, the maintenance dose is administered every 1 month, every 2 months or every 3 months thereafter. In one embodiment, no loading dose is administered, and the composition is administered about once every 1 month, about once every 2 months, about once every 3 months, about once every 4 months, about once every 5 months, or about once every six months.

Another embodiment is a delivery system, comprising a pre-filled single syringe with a composition disclosed herein, in some embodiments together with instructions suitable for using the delivery system to administer the composition to a subject.

In one embodiment, a prefilled syringe system for administration of the compositions disclosed herein, comprising a single syringe containing the composition disclosed herein, is contemplated. In one aspect, the degarelix is dissolved in the biocompatible solvent and the degarelix stays in solution in the solvent. In one aspect, the syringe system is an autoinjector. In one aspect, the syringe system is a reuseable autoinjector, which may be provided with single-use cartridges containing a single dose of a composition of the invention.

In one embodiment, a kit comprising the prefilled syringe system disclosed herein and instructions for use of the prefilled syringe system to administer a composition contained therein is disclosed.

In one embodiment, a product comprising the compositions disclosed herein for use in a method of treating prostate cancer.

In one embodiment, a product comprising the compositions disclosed herein for use in a method of treating CPP is disclosed.

In one embodiment, a product comprising the compositions disclosed herein for use in a method of reducing serum testosterone levels below a castrate level of at least 50 ng/dL is disclosed.

In one embodiment, a product comprising the compositions disclosed herein for use in a method of suppressing ovarian function in a subject with hormone-receptor positive breast cancer is disclosed.

### BRIEF DESCRIPTION OF THE DRAWINGS

FIG. 1 shows the percentage recovery of degarelix from the degarelix/organic solvent formulation samples (formulas 1, 2 and 5 (F1, F2 and F5) (prefilled syringes) stored at 25° C. for 0 months, 1 month, 3 months and 6 months. F1: Degarelix acetate drug substance (DgA-DS)/NMP (65%/35%); F2: DgA-DS/DMSO (65%/35%); F5: DgA-DS/DMSO (60%/35%).

FIG. 2 shows the in vivo degarelix levels in plasma on a linear plot over first 3 days post-injection using a rat model as described in section 13 of Example 1 in the Example section.

FIG. 3 shows the in vivo degarelix levels in plasma on a semi-log plot over 28 days post-injection using a rat model as described in section 13 of Example 1 in the Example section.

FIG. 4 shows the in vivo degarelix levels in plasma over 63 days post-injection using a rat model as described in section 19 of Example 1 in the Example section.

FIG. 5 shows the in vivo degarelix levels in plasma on a semi-log plot over 119 days post-injection using a rat model as described in section 19 of Example 1 in the Example section.

FIG. 6 shows a semi-log view of the in vivo testosterone levels in plasma over 105 days post-injection using a rat model as described in section 19 of Example 1 in the Example section.

FIG. 7 shows the in vivo degarelix levels in plasma on a semi-log plot over 28 days post-injection of degarelix formulations of the invention using a rat model as described in Example 2 in the Example section.

FIG. 8 shows the in vivo testosterone levels in plasma on a semi-log plot over 28 days post-injection of degarelix formulations of the invention using a rat model as described in Example 2 in the Example section.

FIG. 9 shows the in vivo degarelix levels in plasma on a semi-log plot over 63 days post-injection of degarelix formulations of the invention using a rat model as described in Example 2 in the Example section.

FIG. 10 shows the in vivo degarelix levels in plasma on a semi-log plot over 90 days post-injection of degarelix formulations of the invention using a rat model as described in Example 2 in the Example section.

FIG. 11 shows the in vivo testosterone levels in plasma on a semi-log plot over 90 days post-injection of degarelix formulations of the invention using a rat model as described in Example 2 in the Example section.

FIG. 12 shows the in vivo testosterone levels in plasma on a semi-log plot over 140 days post-injection of degarelix formulations of the invention using a rat model as described in Example 2 in the Example section.

FIG. 13 shows the in vivo testosterone levels in plasma on a semi-log plot over 140 days post-injection of degarelix formulations of the invention using a rat model as described in Example 2 in the Example section.

FIG. 14 shows the results of a stability study of acid excipients of various molar ratios in degarelix acetate (DgA)/NMP formulations at 1 month, 3 months and 6 months as described in Example 3 in the Example section. The control (DgA/NMP 35/65 ratio) had no acid additive added.

### DETAILED DESCRIPTION

### Definitions

As used herein, the terms “active pharmaceutical ingredient”, abbreviated as “API”, and “drug” can be used interchangeably and generally refer to a biologically active compound that has therapeutic effects on the body. The “active pharmaceutical ingredient” may refer to an active drug, or a pharmaceutically acceptable salt of an active drug. As used herein, these terms may be used to refer to degarelix or a pharmaceutically acceptable salt thereof.

As used herein, the term “ester” refers to a chemical functional group, C(O)OR′ where R′ represents an alkyl as defined herein. Representative examples include, but are not limited to, methoxycarbonyl, ethoxycarbonyl, propoxycarbonyl, butoxycarbonyl, and the like.

As used herein, the term “antioxidant” refers to compounds which are used to extend the shelf-life of products by preventing or inhibiting the oxidation of active substances and excipients. An antioxidant may react with free radicals thereby blocking or inhibiting free radical chain reactions, or the antioxidant may have a lower redox potential than the active substances and excipients in the formulation. Additionally, or alternatively, a synergist antioxidant may enhance the effects of other antioxidants.

As used herein, the term “biocompatible” means “not harmful to living tissue” or “safe for injection within a human body”.

As used herein, the term “biodegradable” refers to any material that is converted, breaks down, or degrades, under physiological conditions into innocuous or natural byproducts, such as (but not limited to) water, gas, biomass, and/or organic salts, without regard to any specific degradation mechanism or process.

As used herein, the term “liquid” refers to the ability of a composition to undergo deformation under a shearing stress, regardless of the presence or absence of a non-aqueous solvent. “Liquids,” as that term is used herein, may also exhibit viscoelastic behavior, i.e. both viscous and elastic characteristics when undergoing deformation, such as time-dependent and/or hysteretic strain. By way of non-limiting example, viscoelastic materials that are generally flowable but have a partially solid character and/or a plastic- or gel-like character, such as jelly, jam, cake batter or raw pizza dough, and similar materials, are “liquids” as that term is used herein. In some embodiments, materials having a non-zero yield stress that do not deform at stresses below the yield stress, and that are readily deformable without a characteristic of material fracture or rupture at materials above the yield stress, may be “liquids” as that term is used herein.

As used herein, the terms “patient” and “subject” are interchangeable and generally refer to an animal or a human to which a composition disclosed herein is administered or is to be administered.

As used herein, the term “pharmaceutically acceptable salt” refers to a salt of a compound, which possesses the desired pharmacological activity of the parent compound. Such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, lauric acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, pamoic acid, palmitic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

As used herein, the term “solvent” refers to a liquid that dissolves a solid or liquid solute, or to a liquid external phase of a suspension throughout which solid or liquid substances may be suspended or dispersed. The term “biocompatible solvent” may be used interchangeably with the term, “solvent”.

As used herein, the term “co-solvent” refers to a substance added to a solvent to increase or modify the solubility of a solute in the solvent. Accordingly, a co-solvent may increase or decrease solubility of a solute in a primary solvent and/or may impart other desirable characteristics onto a formulation (e.g., the extent of water insolubility of the solvents and co-solvents in a solvent system can impact the desired rate of diffusion into bodily fluids for controlling the rate and scope of degarelix API gelation; or solvent/co-solvents can control the viscosity of the compositions of the invention, which aids in preparing and administering the extended release composition to a subject).

As used herein, the term “solvent system” refers to the combination of at least one biocompatible solvent as described herein which may optionally include at least one co-solvent.

As used herein, the term “solubilizer” refers to a compound that increases the solubility of another substance.

As used herein, the term “surfactant” refers to a compound that lowers the surface tension between two liquids, between a gas and a liquid, or between a liquid and a solid. For example, a surfactant can act as a wetting agent, which aids in dispersing an active pharmaceutical ingredient in a liquid vehicle, or as a solubilizer.

As used herein, the term “therapeutically effective amount” means the amount of a compound or drug product that, when administered to a patient for treating a disease and/or treating or preventing one or more symptoms of a disease, is sufficient to affect such treatment or prevention for the disease. The “therapeutically effective amount” can vary depending on, for example, the compound, disease progression, the disease or condition to be treated, whether the therapy is an adjuvant therapy or a primary or curative therapy, and/or the age, weight, etc., of the patient to be treated.

As used herein, the terms “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C”, “A, B, and/or C”, and “A, B, or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

As used herein, the term “depot” (which may also be referred herein to as “fibrils” or “gel”) refers to the degarelix composition/formulation disclosed herein once exposed to an aqueous environment (e.g., including a physiological environment in a body of an animal) in which the degarelix API self-aggregates and creates a drug reservoir from which the degarelix is released over an extended period of time.

Unless otherwise specified, various amounts of API and solvents and co-solvents are reported in weight percentages of the solvent system or pharmaceutical composition.

Every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include both terminal values as well as each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

Reference will now be made in detail to particular embodiments of compounds, formulations and methods. The disclosed embodiments are not intended to be limiting of the claims.

Degarelix is a decapeptide selective gonadotropin-releasing hormone (GnRH) receptor antagonist (blocker) that competitively and reversibly binds to GnRH receptors in the pituitary. This results in a significant reduction of follicle-stimulating hormone (FSH) and luteinizing hormone (LH), thus reducing testosterone levels in males, or estrogen levels in females. In one embodiment of the invention, degarelix is indicated for LH suppression or testosterone suppression in adult male patients with advanced hormone-dependent prostate cancer in whom androgen deprivation is warranted. Degarelix formulations of the present invention are also useful for LH suppression or estrogen (e.g., estradiol) suppression in adult female patients with pre-menopausal or perimenopausal breast cancer, and are also useful for conditions including, but not limited to, endometriosis. Degarelix formulations of the present invention are also useful for LH suppression or sex hormone suppression (testosterone or estrogen, depending on whether the patient is male or female) in children with central precocious puberty (CPP). There is an unmet need for a long-acting injectable formulation of degarelix that (1) is easy to prepare and administer (e.g., by a health care provider); (2) provides sustained or extended release of degarelix for more than a month after a single dose, and in one embodiment, for at least three months or longer; and (3) is administered in a total injection volume of about 2.5 mL or less, or about 2 mL or less.

Disclosed herein is a long-acting, injectable composition or formulation (used interchangeably and which may also be referred to as a “drug product” “pharmaceutical product” “product” or “finished drug product”) that comprises degarelix or a pharmaceutically acceptable salt thereof, in an organic solvent extended-release injectable formulation. These formulations do not comprise a biodegradable polymer. The formulation comprises a biocompatible, water-miscible solvent and can optionally further include one or more additives as disclosed herein. The degarelix composition/formulation disclosed herein is a flowable solution or suspension that upon delivery (i.e., injection) into an aqueous environment (e.g., the body of a human), the water-miscible solvent exchanges with surrounding aqueous body fluids, which results in the formation of a depot or fibrils or gelation of degarelix that acts as a drug reservoir for extended-release of degarelix peptide (i.e. release of the drug over an extended period of time).

The composition/formulations disclosed herein provide sustained or extended release of degarelix or a pharmaceutically acceptable salt thereof for more than a month when injected at low volumes (e.g., <2.5 mL or ≤2 mL) via subcutaneous injection. The long-acting injectable compositions suitable for use in the methods of this disclosure, which may also be referred to as pharmaceutical compositions or formulations, extended-release compositions or formulations, or controlled release compositions or formulations, provide a biodegradable or bioerodible in situ formed depot of degarelix or a pharmaceutically acceptable salt thereof in a subject, from which the degarelix or a pharmaceutically acceptable salt thereof, is released over a period of one month or longer. The composition/formulation can be supplied in a prefilled single syringe with injection volumes of less than or equal to 2.5 mL, less than or equal to 2.4 mL, less than or equal to 2.3 mL, less than or equal to 2.2 mL, less than or equal to 2.1 mL, less than or equal to 2.0 mL, less than or equal to 1.9 mL, less than or equal to 1.8 mL, less than or equal to 1.7 mL, less than or equal to 1.6 mL, or less than equal to 1.5 mL and is suitable for subcutaneous injection. This composition solves the problem of the multiple, high-volume injections provided by the current degarelix product on the market.

After administration of the compositions disclosed herein, degarelix or a pharmaceutically acceptable salt thereof is released from the depot over a period of at least about 1 month, at least about 1.5 months, at least about 2 months, at least about 2.5 months, at least about 3 months, at least about 3.5 months, at least about 4 months, at least about 4.5 months, at least about 5 months, or at least about 6 months. The duration of release from the depot can depend on one or a number of factors including, for example, the solvent, the amounts of the components in the composition, optional additives in the composition, and combinations of any of the foregoing, or other factors.

### Active Pharmaceutical Ingredient (API)

The compositions/formulations disclosed herein comprise degarelix or a pharmaceutically acceptable salt thereof in solvent-based drug delivery system. The pharmaceutically acceptable salts of degarelix include, but are not limited to, degarelix acetate, degarelix citrate, degarelix pamoate, and degarelix mesylate. “Degarelix API” (whether capitalized or not) as used herein refers to degarelix or any pharmaceutically acceptable salt thereof.

Generally, the disclosed compositions comprise a biocompatible solvent, and degarelix or a pharmaceutically acceptable salt thereof. The pharmaceutical compositions are administered to a patient by subcutaneous injection as a liquid or gel wherein a solid, semi-solid, or liquid depot, consisting of fibrils that form a gel of degarelix in situ upon dissipation of the solvent. The depot so formed releases degarelix or a pharmaceutically acceptable salt thereof, in a controlled, or extended, release manner.

The concentration of the degarelix or a pharmaceutically acceptable salt thereof, in the disclosed compositions can vary and may range from 1% to 50% by weight of the composition, including any whole number percent to any other whole number percent within the range of from about 1 percent to about 50 percent by weight. The concentration of the degarelix or pharmaceutically acceptable salt thereof in the composition may be about 5% by weight of the composition, or about 10% by weight of the composition, or about 15% by weight of the composition, or about 20% by weight of the composition, or about 21% by weight of the composition, or about 22% by weight of the composition, or about 23% by weight of the composition, or about 24% by weight of the composition, or about 25% by weight of the composition, or about 26% by weight of the composition, or about 27% by weight of the composition, or about 28% by weight of the composition, or about 29% by weight of the composition, or about 30% by weight of the composition, or about 31% by weight of the composition, or about 32% by weight of the composition, or about 33% by weight of the composition, or about 34% by weight of the composition, or about 35% by weight of the composition, or about 36% by weight of the composition, or about 37% by weight of the composition, or about 38% by weight of the composition, or about % by weight of the composition, or about 40% by weight of the composition, or about 45% by weight of the composition, or about 50% by weight of the composition. In other embodiments, the amount of degarelix API in compositions of the invention may range from any tenth of a percent to any other tenth of a percent within the range of from about 1% to about 50% by weight.

In some aspects, the amount of degarelix API in a composition of the invention is referenced with respect to the amount of a pharmaceutically acceptable salt of degarelix, such as degarelix acetate or other salt, in the composition. In some aspects, the amount of degarelix API in a composition of the invention is referenced with respect to the amount of degarelix free base equivalent in the composition (which may also simply be referred to as the amount of degarelix in the composition). By way of example, determination of the dose amount of degarelix API to be administered to a subject may be calculated based on the amount of degarelix free base equivalent in the composition rather than the amount of degarelix salt in order to account for differences in purity, water, or acetic acid content (e.g., in the case of degarelix acetate), among various API lots. Calculation of the free base equivalent of degarelix in a composition is well understood within the art. For example, the percentage (by weight) of degarelix as free base in a composition where the degarelix API is degarelix acetate can be calculated in a simple formula such as: 100−(wt % drug substance water content−wt % drug substance acetic acid content)*degarelix purity by assay (anhydrous/acid free base)*wt % degarelix acetate in the composition. The free base equivalent may also be provided from the USP Monograph for a drug substance.

In one aspect, the amount of degarelix or a pharmaceutically acceptable salt thereof (degarelix API) in a composition of the invention is from about 30 wt % to about 35 wt % degarelix free base equivalent. In one aspect, the amount of degarelix API in the pharmaceutical composition is from about 31 wt % to about 34 wt % degarelix free base equivalent. In one aspect, the amount of degarelix API in the pharmaceutical composition is from about 31 wt % to about 33 wt % degarelix free base equivalent. In one aspect, the amount of degarelix API in the pharmaceutical composition is from about 31 wt % to about 32 wt % degarelix free base equivalent. In one aspect, the amount of degarelix API in the pharmaceutical composition is from about 20 wt % to about 23 wt % degarelix free base equivalent. In one aspect, the amount of degarelix API in the pharmaceutical composition is from about 20 wt % to about 22 wt % degarelix free base equivalent. In one aspect, the amount of degarelix API in the pharmaceutical composition is from about 21 wt % to about 23 wt % degarelix free base equivalent. In one aspect, degarelix API in the pharmaceutical composition is from about 21 wt % to about 22 wt % degarelix free base equivalent. In some aspects, when a target dose to be administered to a subject is calculated based on degarelix free base equivalent in the composition, the percent by weight of solvent, and the percent by weight of additive if included, may be modified accordingly.

### Solvents

Any suitable water-miscible solvent can be employed, provided the solvent is miscible to dispersible in aqueous medium or body fluid. Examples of suitable solvents are disclosed, e.g., in Aldrich Handbook of Fine Chemicals and Laboratory Equipment, Milwaukee, Wis. (2000); and in U.S. Pat. Nos. 5,324,519; 4,938,763; 5,702,716; 5,744,153; and 5,990,194. In various aspects, the solvent is able to diffuse into body fluid so that the flowable composition coagulates or solidifies. Solvents and co-solvents that may be used in the disclosed compositions are preferably biocompatible, non-toxic, solvents, which may be either hydrophilic or hydrophobic solvents, or may be a combination of hydrophilic solvents, hydrophobic solvents or hydrophilic and hydrophobic solvents, depending upon the desired release profile and the solubility of the degarelix API in the composition. In one aspect, the solvent and/or co-solvent is an organic solvent. In still another aspect, the solvent and/or co-solvents is a polar aprotic solvent.

Suitable solvents and co-solvents may comprise one or more solvents selected from the group consisting of amides, acids, alcohols, esters of monobasic acids, ether alcohols, sulfoxides, lactones, polyhydroxy alcohols, esters of polyhydroxy alcohols, ketones, and ethers. By way of non-limiting example, the solvents or cosolvents may comprise at least one of N-methyl-2-pyrrolidone (NMP), dimethyl sulfoxide (DMSO), dimethylacetamide (DMA), acetone, benzyl benzoate (BnBzO), polyethylene glycol 15 hydroxystearate, ethyl acetate, glycofurol, N-hydroxyethyl-2-pyrrolidone, polyethylene glycol (PEG), benzyl alcohol (BzOH), propylene carbonate (PC), propylene glycol, 2-pyrrolidone, α-tocopherol, triacetin, tributyl citrate, acetyl tributyl citrate, acetyl triethyl citrate, triethyl citrate, esters thereof, and combinations thereof.

In aspects of the invention, a suitable solvent is selected from N-methyl-2-pyrrolidone (NMP) and/or dimethyl sulfoxide (DMSO). In aspects of the invention, a suitable co-solvent includes benzyl alcohol (BzOH).

Also disclosed herein, the degarelix or pharmaceutically acceptable salt thereof is preferably substantially or fully dissolved in the biocompatible solvent or solvent system.

The biocompatible solvent, or combination or mixture of solvents and/or co-solvents used in a composition of the invention, will generally comprise between about 50 wt % and about 99 wt % of the final formulation, or between about 30 wt % and about 69 wt % of the final formulation, or between about 40 wt % and about 59 wt % of the final formulation, or alternatively the solvent or combination or mixture of solvents and/or co-solvents can range from any whole number percentage by weight of the formulation to any other whole number percentage by weight of the formulation between about 50 wt % and about 99 wt %. If the formulation includes the optional co-solvent and/or additive, while the ratio of solvent to degarelix API may be the same, the wt % of each in the final formulation may be altered due to the presence of the co-solvent and/or additive.

### Additives

Optionally, the pharmaceutical compositions disclosed herein may comprise various additives (which may also be referred to as “excipients”) to improve the stability, the injectability, or/and other properties of the composition, including reduction of pain or inflammation upon injection of the composition. For example, the pharmaceutical compositions may comprise one or more of antioxidants, chelating agents, surfactants, co-solvents (also discussed above), stabilizers, complexing agents, antioxidants, and solubilizers.

In some embodiments, the pharmaceutical compositions may comprise one or more solubilizers to increases the solubility of one or more other component of the composition. Solubilizers useful in the disclosed compositions include any solubilizer useful for parenteral injection, and include, but are not limited to, surfactants which lower the surface tension between two liquids, between a gas and a liquid, or between a liquid and a solid and other solubilizers. Examples of suitable solubilizers and/or surfactants for use in the invention include, but are not limited to, polysorbate20, polysorbate80, Poloxamer 188, sorbitan trioleate, lecithin (e.g., soya or egg), Vitamin E TPGS, sugar based esters or ethers (e.g., sugar acid esters of fatty alcohols or sugar alcohol esters of fatty acids, including, but not limited to, sucrose cocoate, sucrose stearate, sucrose laurate, etc.), amino acid-based solubility enhancers (e.g., proline, arginine, DL-methionine), protein-based solubility enhancers (e.g., hydrophobins), and others.

In some embodiments, the pharmaceutical compositions may comprise one or more antioxidants to inhibit oxidation of the API and improve the stability of the formulation. Examples of suitable antioxidants for use in the invention include, but are not limited to, citric acid, methanesulfonic acid, ascorbic acid, ethylenediaminotetraacetic acid (EDTA), mercaptoethanol, sodium metabisulfite butylated hydroxyanisole, butylated hydroxyquinone, butylhydroxyanisol, hydroxycoumarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine.

In some embodiments, the pharmaceutical compositions may comprise one or more complexing agents to inhibit oxidation and/or degradation of the API and improve the stability of the formulation. Examples of suitable complexing agents for use in the invention include, but are not limited to, ethylenediaminotetraacetic acid (EDTA), divalent metal salts (ZnCl₂, MgCl₂, CaCl₂)), nitrilotriacetic acid, n-hydroxyethylethylenediaminetriacetic acid (HEDTA), ethylene glycol-bis(β-aminoethyl ether)-N,N,N′,N′-tetraacetic acid (EGTA) as well as several simple organic acids, such as polycarboxylic acid (citric acid), hydrochloric acid, sulfuric acid, pamoic acid, and palmitic acid.

In some embodiments, the pharmaceutical compositions may comprise one or more release modifying additives.

In some embodiments, the pharmaceutical compositions may comprise one or more stabilizing agents to prevent drug degradation (physical or chemical) and improve the stability of the formulation and increase shelf life. Examples include, but are not limited to: surfactants (e.g., Polysorbate20, Polysorbate 80, poloxamer 188), complexing agents (e.g., divalent metal salts), acid additives (e.g., acetic acid (AcOH), citric acid, succinic acid, methane sulfonic acid, sulfuric acid, hydrochloric acid (HCl), pamoic acid, palmitic acid); and alcohols (e.g., benzyl alcohol).

For example, in some embodiments, an acid additive is optionally included in a pharmaceutical composition of the invention as an additional excipient (i.e., in addition to the degarelix API and solvent and/or co-solvent). The acid additive as an excipient provides additional acid in the compositions of the invention, in addition to any acid that is provided by the pharmaceutical salt of the API itself (e.g., the acid content in degarelix acetate drug substance). Such an acid excipient may improve the stability of the formulation and increase shelf life by, for example, reducing the level of impurities that develop in the formulation over time prior to use and/or decreasing the amount of aggregation of the degarelix API over time prior to use. Acid additives can include, but are not limited to, acetic acid (AcOH), citric acid, succinic acid, methane sulfonic acid, sulfuric acid, hydrochloric acid (HCl), pamoic acid, palmitic acid, hydrobromic acid, nitric acid, chromic acid, trifluoroethanes sulfonic acid, trichloroacetic acid, dichloroacetic acid, bromoacetic acid, chloroacetic acid, cyanoacetic acid, 2-chloropropanoic acid, 4-cyanobutanoic acid, perchloric acid, phosphoric acid, hydrogen iodide, and combinations thereof. In one aspect, the acid additive is acetic acid, citric acid and/or succinic acid.

In aspects, the concentration of the acid additive as an excipient in the disclosed compositions can vary and may range from about 0.1% to about 10% by weight of the composition, including any increment of 0.1 percent within the range of from about 0.1 percent to about 10 percent by weight. In one aspect, the concentration of the acid additive as an excipient in the disclosed compositions ranges from about 0.1 wt % to about 9 wt %; or from about 0.1 wt % to about 8 wt %; or from about 0.1 wt % to about 7 wt %; or from about 0.1 wt % to about 6 wt %; or from about 0.1 wt % to about 5 wt %; or from about 0.1 wt % to about 4 wt %; or from about 0.1 wt % to about 3 wt %; or from about 0.1 wt % to about 2 wt %; or from about 0.1 wt % to about 2 wt %; or from about 0.5 wt % to about 10 wt %; or from about 0.5 wt % to about 9 wt %; or from about 0.5 wt % to about 8 wt %; or from about 0.5 wt % to about 7 wt %; or from about 0.5 wt % to about 6 wt %; or from about 0.5 wt % to about 5 wt %; or from about 0.5 wt % to about 4 wt %; or from about 0.5 wt % to about 3 wt %; or from about 0.5 wt % to about 2 wt %; or from about 1 wt % to about 10 wt %; or from about 1 wt % to about 10 wt %; or from about 1 wt % to about 9 wt %; or from about 1 wt % to about 8 wt % or from about 1 wt % to about 7 wt % or from about 1 wt % to about 6 wt %; or from about 1 wt % to about 5 wt %; or from about 1 wt % to about 4 wt %; or from about 1 wt % to about 3 wt %; or from about 1 wt % to about 2 wt %.

In aspects the amount of the acid additive as an excipient in the disclosed compositions can be expressed as a molar ratio of degarelix API (drug substance prior to addition to the formulation) to acid additive. In aspects, the molar ratio of degarelix API (calculated as free base) to acid additive in the formulation is 1:1, or 1:2, or 1:3, or 1:4, or 1:5, or 1:6. In another aspect, the molar ratio of degarelix API (calculated as free base) to the total amount of acid in the formulation (contributed by the degarelix API salt form and acid additive, if any), is 1:1, or 1:2, or 1:3, or 1:4, or 1:5, or 1:6, or 1:7, or 1:8, or 1:9.

In aspects, the amount of the acid in the composition of the invention can alternatively be expressed as a total amount of acid contributed by all components in the formulation, i.e., from the degarelix API salt form and any acid additive as an excipient. In aspects, the total amount of acid in the composition (as a wt % of the composition) ranges from about 1% to about 10%; or from about 1.5% to about 10%; or from about 2.0% to about 10%; or from about 2.5% to about 10%; or from about 3.0% to about 10%; or from about 3.5% to about 10%; or from about 4.0% to about 10%; or from about 4.5% to about 10%; or from about 5.0% to about 10%; or from about 5.5% to about 10%; or from about 6.0% to about 10%; or from about 6.5% to about 10%; or from about 7.0% to about 10%; or from about 7.5% to about 10%; or from about 8.0% to about 10%; or from about 8.5% to about 10%; or from about 9.0% to about 10%; or from about 9.5% to about 10%, or from about 1.0% to about 7.0%, or from about 1.0% to about 6.0%, or from about 1.0% to about 5.0%, or from about 1.0% to about 4.0%, or from about 1.0% to about 3.0%, or from about 1.0% to about 2.0%, or in any increment of 0.1% between 1% and 10%.

In aspects, the amount of acid in the degarelix drug substance (degarelix API itself, not as an excipient) prior to addition to the composition or formulation ranges from about 0 wt % to about 10 wt %, 1 wt % to about 10 wt %, or from about 2 wt % to about 10 wt %, or from about 3 wt % to about 10 wt %, or from about 3.5 wt % to about 10 wt %, or from about 4 wt % to about 10 wt %, or from about 4.5 wt % to about 10 wt %, or from about 5 wt % to about 10 wt %, or from about 5.5 wt % to about 10 wt %, or from about 6 wt % to about 10 wt %, or from about 6.5 wt % to about 10 wt %, or from about 7 wt % to about 10 wt %, or from about 7.5 wt % to about 10 wt %, or from about 8 wt % to about 10 wt %, or from about 8.5 wt % to about 10 wt %, or from about 9 wt % to about 10 wt %, or from about 9.5 wt % to about 10 wt %.

In still other aspects, the composition further comprises an additive (excipient) that is an alcohol, which can serve as a co-solvent, and which may also provide additional properties to the composition, including but not limited to, improved formulation stability, improved injectability, and/or decreased pain, inflammation or irritation upon injection into a body. In one aspect, formulations of the invention optionally contain benzyl alcohol as an additive/excipient. In aspects, the concentration of the alcohol additive as an excipient in the disclosed compositions can vary and may range from about 1% to about 30% by weight of the composition, including any increment of 1 percent within the range of from about 1 percent to about 30 percent by weight of the composition. In one aspect, the concentration of the alcohol additive as an excipient in the disclosed compositions ranges from about 1 wt % to about 30 wt %; or from about 5 wt % to about 30 wt %; or from about 10 wt % to about 30 wt %; or from about 11 wt % to about 30 wt %; or from about 12 wt % to about 30 wt %; or from about 13 wt % to about 30 wt %; or from about 14 wt % to about 30 wt %; or from about 15 wt % to about 30 wt %; or from about 16 wt % to about 30 wt %; or from about 17 wt % to about 30 wt %; or from about 18 wt % to about 30 wt %; or from about 19 wt % to about 30 wt %; or from about 20 wt % to about 30 wt %; or from about 21 wt % to about 30 wt %; or from about 22 wt % to about 30 wt %; or from about 23 wt % to about 30 wt %; or from about 24 wt % to about 30 wt %; or from about 25 wt % to about 30 wt %; or from about 26 wt % to about 30 wt %; or from about 27 wt % to about 30 wt %; or from about 28 wt % to about 30 wt %; or from about 29 wt % to about 30 wt %.

### Compositions

In various aspects, the present disclosure provides long acting, injectable pharmaceutical compositions comprising degarelix or pharmaceutically acceptable salt thereof, a biocompatible solvent, and optionally one or more additives. All such compositions are contemplated for administration to a subject to treat a disease or condition for which administration of a GnRH agonist or GnRH antagonist may be useful. In various aspects, the compositions of the invention are contemplated for use to treat prostate cancer, including advanced prostate cancer; to treat central precocious puberty (CPP); to treat pre-menopausal or peri-menopausal breast cancer; or to treat other conditions, including, but not limited to endometriosis. Further, such compositions are contemplated for administration to a subject to reduce luteinizing hormone (LH) levels in a subject, and in males, to reduce serum testosterone levels, and in females, to reduce serum estrogen levels.

Depending upon the other components in the composition, the degarelix or a pharmaceutically acceptable salt thereof, in the disclosed compositions may form a monophasic mixture (e.g., a solution) or a biphasic mixture (e.g., a suspension or a dispersion) within the composition. Accordingly, the extended release compositions comprising the degarelix or pharmaceutically acceptable salt thereof, according to the present invention may appropriately be either a “solution” or a “dispersion” or a “suspension” of the degarelix or pharmaceutically acceptable salt thereof, in the biocompatible solvent. Preferably, the degarelix or pharmaceutically acceptable salt thereof is in solution in the biocompatible solvent and is thus uniphase. More preferably, the degarelix API is a stable solution and stays in solution in the biocompatible solvent and thus no reconstitution or mixing is needed to maintain the stable solution.

In some embodiments, the present disclosure provides a pharmaceutical composition comprising about 20 wt % to about 40 wt % of degarelix acetate or degarelix citrate; and about 60 wt % to about 80 wt % of N-methyl-2-pyrrolidone (NMP) or dimethyl sulfoxide (DMSO).

In some embodiments, the present disclosure provides a pharmaceutical composition comprising about 25 wt % to about 45 wt % degarelix acetate or degarelix citrate and about 55 wt % to about 75 wt % of NMP or DMSO.

In some embodiments, the present disclosure provides a pharmaceutical composition comprising degarelix API in an amount from about 20 wt % to about 45 wt % degarelix free base equivalent. In some aspects, the present disclosure provides a pharmaceutical composition comprising degarelix API in an amount from about 20 wt % to about 40 wt % degarelix free base equivalent, or from about 20 wt % to about 35 wt % degarelix free base equivalent, or from about 20 wt % to about 34 wt % degarelix free base equivalent, or from about 20 wt % to about 33 wt % degarelix free base equivalent, or from about 20 wt % to about 32 wt % degarelix free base equivalent. In some embodiments, the present disclosure provides a pharmaceutical composition comprising degarelix API in an amount from about 30 wt % to about 35 wt % degarelix free base equivalent. In one aspect, the amount of degarelix API in the pharmaceutical composition is from about 31 wt % to about 34 wt % degarelix free base equivalent. In one aspect, the amount of degarelix API in the pharmaceutical composition is from about 31 wt % to about 33 wt % degarelix free base equivalent. In one aspect, the amount of degarelix API in the pharmaceutical composition is from about 31 wt % to about 32 wt % degarelix free base equivalent.

In one aspect, the amount of degarelix API in the pharmaceutical composition is from about 20 wt % to about 23 wt % degarelix free base equivalent. In one aspect, the amount of degarelix API in the pharmaceutical composition is from about 20 wt % to about 22 wt % degarelix free base equivalent. In one aspect, the amount of degarelix API in the pharmaceutical composition is from about 21 wt % to about 23 wt % degarelix free base equivalent. In one aspect, degarelix API in the pharmaceutical composition is from about 21 wt % to about 22 wt % degarelix free base equivalent. In some aspects, when a target dose to be administered to a subject is calculated based on degarelix free base equivalent in the composition, the percent by weight of solvent, and the percent by weight of additive if included, may be modified accordingly.

In some embodiments, as described in more detail above, the present disclosure provides a pharmaceutical composition that further includes an acid additive as an excipient. In one aspect, the composition further comprises from about 0.1 wt % to about 10.0 wt % of an acid additive, or from about 1.0 wt % to about 6.0 wt % of an acid additive, or from about 1.0 wt % to about 5.0 wt % of an acid additive, or from about 1.0 wt % to about 4.0 wt % of an acid additive, or from about 1.0 wt % to about 3.0 wt % of an acid additive, or from about 1.0 wt % to about 2.0 wt % of an acid additive. In one aspect, the acid is acetic acid, citric acid or succinic acid.

In still other embodiments, present disclosure provides a pharmaceutical composition that further includes benzyl alcohol as an additional excipient. In one aspect, the composition further comprises from about 5.0 wt % to about 30 wt % of benzyl alcohol (BnOH).

In some embodiments, the present disclosure provides a pharmaceutical composition comprising about 35 wt % of degarelix acetate and about 65 wt % of N-methyl-2-pyrrolidone (NMP).

In some embodiments, the present disclosure provides a pharmaceutical composition comprising about 35 wt % of degarelix acetate and about 65 wt % of DMSO.

In some embodiments, the present disclosure provides a pharmaceutical composition comprising about 20-40 wt % of degarelix acetate; about 55-70 wt % of NMP; and about 5-10 wt % of benzyl alcohol (BnOH).

In some embodiments, the present disclosure provides a pharmaceutical composition comprising about 35 wt % of degarelix citrate; and about 65 wt % of NMP.

In some embodiments, the present disclosure provides a pharmaceutical composition comprising about 35 wt % of degarelix acetate; about 62 to 64 wt % NMP; and about 1 to 5 wt % AcOH.

In some embodiments, the present disclosure provides a pharmaceutical composition comprising about 35 wt % degarelix acetate; about 62 to 64 wt % NMP; and 1 to 3 wt % AcOH.

In some embodiments, the present disclosure provides a pharmaceutical composition comprising about 35 wt % of degarelix acetate; about 62.8 wt % NMP; and about 2.2 wt % AcOH.

In some embodiments, the present disclosure provides a pharmaceutic composition comprising about 24 wt % of degarelix acetate; about 50 to 60 wt % NMP; and about 16 to 26 wt % BnOH.

In some embodiments, the present disclosure provides a pharmaceutic composition comprising about 24 wt % of degarelix acetate; about 54 to 58 wt % NMP; and about 18 to 22 wt % BnOH.

In some embodiments, the present disclosure provides a pharmaceutic composition comprising about 24 wt % of degarelix acetate; about 56 wt % NMP; and about 20 wt % BnOH.

In some embodiments, the present disclosure provides a pharmaceutic composition comprising about 24 wt % of degarelix acetate; about 66 to 75 wt % NMP; and about 1 to 10 wt % AcOH.

In some embodiments, the present disclosure provides a pharmaceutic composition comprising about 24 wt % of degarelix acetate; about 70 to 75 wt % NMP; and about 1 to 6 wt % AcOH.

In some embodiments, the present disclosure provides a pharmaceutic composition comprising about 24 wt % of degarelix acetate; about 73.3 wt % NMP; and about 1.7 wt % AcOH.

### Treatment Methods, Uses and Administration

The methods of this disclosure are used in the treatment of diseases or conditions including prostate cancer, including advanced prostate cancer. Still further, the methods of this disclosure are used in reducing serum testosterone levels below a castrate level of at least 50 ng/dL, or below 20 ng/dL or below 10 ng/dL.

Further, the methods of this disclosure are used in suppressing ovarian function in a subject with hormone-receptor positive breast cancer. In one aspect, the hormone receptor positive breast cancer is pre-menopausal breast cancer. In one aspect, the hormone receptor positive breast cancer is peri-menopausal breast cancer. In one aspect, the hormone receptor positive breast cancer is estrogen receptor (ER) positive breast cancer. In one aspect, the compositions disclosed herein suppress a subject's estradiol (E2) production to a level less than about 20 pg/ml, less than about 15 pg/mL, less than about 10 pg/mL, less than about 5 pg/mL, less than about 4 pg/ml, less than about 3 pg/mL, or less than about 2 pg/mL. In a preferred aspect, the E2 production level is reduced to about 2.7 pg/mL. In still another aspect, the compositions disclosed herein suppresses the breast cancer subject's follicle stimulating hormone (FSH) to a level less than about 40 IU/L. In yet another aspect, the compositions disclosed herein suppresses the breast cancer subject's luteinizing hormone (LH) to a level less than about 4 IU/L.

Still further, the methods of this disclosure are used in treating endometriosis.

The methods of this disclosure are used in the treatment of central precocious puberty (CPP). CPP is defined by early sexual development prompted by production and release of gonadotropins and/or sex steroids from normal endogenous sources including the hypothalamus or pituitary. Aberrations in gonadotropin and/or sex hormone concentration levels in children with CPP can result from various sources, including, but not limited to, physical injury, infection, genetic disease, or associated tumors. CPP caused by a genetic or undetermined pathology is classified to be idiopathic in nature, while CPP caused by a central nervous system (CNS) tumor and/or lesion is classified as organic in nature. CPP is accompanied by advanced bone age, accelerated growth velocity, and Hypothalamic-Pituitary-Gonadal-axis activation. In one aspect, the compositions disclosed herein reduce a subject having CPP blood serum LH concentration to a pre-pubertal concentration levels of <4 IU/L.

The methods comprise subcutaneously or intramuscular administering a disclosed long-acting injectable composition to a subject/patient suffering from prostate cancer, advance prostate cancer, CPP, pre-menopausal breast cancer, post-menopausal breast cancer, as well as those subjects in head of reducing serum testosterone levels and/or LH levels. Upon injection of the pharmaceutical composition into the body and contact of the composition with a bodily fluid, the solvent dissipates and self-aggregation or gelation of degarelix occurs, forming a drug reservoir or depot. The resulting depot will release the degarelix or a pharmaceutically acceptable form thereof, over a desired extended time period. In various embodiments, the degarelix or a pharmaceutically acceptable salt thereof is released into a subject/patient, for example for a period of at least about 30 days or longer, at least about 60 days or longer, at least about 90 days or longer, at least about 120 days or longer, at least about 150 days or longer, or 180 days or longer. In still other embodiments, the degarelix or a pharmaceutically acceptable salt thereof is released into a subject/patient, for example for a period of at least about one month or longer, at least about two months or longer, at least about three months or longer, at least about four months or longer, at least about five month or longer, or six months or longer.

The long-acting composition may be administered to the patient/subject once about every 30 days, once about every 60 days, once about every 90 days, once about every 120 days, once about every 150 days or once about every 180 days. In another aspect, the long-acting composition may be administered to the patient/subject once about every 1 month, once about every 2 months, once about every 3 months, once about every 4 months, once about every 5 months or about once every 6 months. In a preferred aspect, the composition is administered once about every 3 months. According to the present invention, for clarity, a “month” can have between 28 and 31 days.

The amount of degarelix or a pharmaceutically acceptable salt thereof, that will be effective in the treatment or reduction of the above diseases or conditions, will depend on the nature/severity of the condition or symptom. In vitro or in vivo assays may optionally be employed to help identify optimal dosage ranges. The amount of degarelix or a pharmaceutically acceptable salt thereof, administered will, of course, be dependent on, among other factors, the subject being treated, the age/weight of the subject, the severity of the affliction, the manner of administration and the judgment of the prescribing physician.

The dose amount of degarelix or a pharmaceutically acceptable salt thereof for treating prostate cancer and/or advanced prostate cancer, in the composition is administered in a dose amount of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 210 mg, about 220 mg, about 225, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 310 mg, about 320 mg, about 330 mg, about 340 mg, about 350 mg, about 360 mg, about 370 mg, about 380 mg, about 390 mg, about 400, about 410 mg, about 420 mg, about 430 mg, about 440 mg, about 450 mg, about 460 mg, about 470 mg, about 480 mg, about 490 mg, or about 500 mg. In one aspect, a single dose may be administered in one injection. In still another aspect, a single dose may be administered in more than one injection.

In one aspect, the disclosed compositions are to be administered to a subject/patient once in a dosing period with varying durations between dosing (e.g., one month, 2 months, 3 months, 4 months, 5 months, or 6 months). In one aspect, the disclosed compositions are to be administered to a subject/patient in an initial loading dose followed by one or more maintenance doses of the disclosed compositions. Dosing may be provided alone or in combination with other drugs and may continue as long as required for effective treatment of the disease state or disorder. In some embodiments, the disclosed compositions are formulated to provide doses between about 40 mg-about 500 mg. In these embodiments, the composition is delivered in a total volume, or per injection volume, not to exceed 2.5 mL or not to exceed 2 mL. In some embodiments, the injection volume is about 2.5 mL or less, about 2.4 mL or less, about 2.3 mL or less, about 2.2 mL or less, about 2.1 mL or less, about 2.0 mL or less, about 1.9 mL or less, about 1.8 mL or less, about 1.7 mL or less, about 1.6 mL or less, about 1.5 mL or less, about 1.4 mL or less, about 1.3 mL or less, about 1.2 mL or less, about 1.1 mL or less, about 1 mL or less, about 0.75 mL or less, about 0.5 mL or less, or about 0.375 mL or less. In some embodiments, the injection volume is about 0.375 ml, about 0.5 mL, about 0.75 mL, about 1 mL, about 1.5 mL, or about 1.75 mL, or about 2 mL, about 2.2 mL or about 2.5 mL.

In some embodiments, the composition is terminally sterilized such as by e-beam or Gamma irradiation or X-ray. In yet another aspect, the composition is sterile filtered.

In some embodiments, the long-acting composition is administered as a monotherapy to patients. The therapeutic methods of this embodiment may reduce or eliminate one or more symptoms of the disease and/or condition disclosed herein. In other embodiments, the long-acting composition may be administered as a combination therapy, such as with chemotherapeutics, radiation therapy, surgery, endocrine therapies such as selective estrogen receptor modulators (SERMs; such as Tamoxifen, Toremifene, Raloxifene, Ospemifene, and Bazedoxifene), selective estrogen receptor degraders (SERDs; such as fulvestrant), aromatase inhibitors (AIs; such as anastrozole, letrozole, exemestane, vorozole, formestane, and fadrozole); mammalian target of rapamycin (mTOR) inhibitors; such as temsirolimus, sirolimus, everolimus, and ridaforolimus); Phosphatidylinositol 3-kinases inhibitors (PI-3 kinase or PI3K; such as alpelisib, idelalisib, and buparlisib); cyclin-dependent kinases 4 and 6 inhibitors (CDK4/6 inhibitors; such as abemaciclib, palbociclib, and ribociclib); LHRH agonists (such as leuprolide, gonadorelin, goserelin, histrelin, nafarelin, buserelin, and triptorelin and their pharmaceutically acceptable salts thereof), immuno-therapy, and gene therapy.

The disclosed long-acting composition may be provided as a part of a delivery system comprising a syringe system, wherein the composition or formulation is contained within a syringe. In some embodiments, the syringe is a pre-filled syringe system containing the disclosed composition as a single dose or as multiple doses. The syringe may contain a single dose or multiple doses of a composition/formulation of the invention. In one aspect of the pre-filled syringe system, the syringe is a mixing syringe (e.g., a syringe that provides a mechanism for mixing the formulation contained therein, as needed). In one aspect, the pre-filled syringe is a single chamber syringe, where the composition contained therein is a stable, uniphase or substantially uniphase composition that does not require mixing prior to injection. In one aspect, the syringe system is an autoinjector. In one aspect, the syringe system is a reuseable autoinjector, which may be provided with single-use cartridges containing a single dose of a composition of the invention. In one aspect, the syringe system may be referred to as a container. A container may also be a syringe and a vial containing a single dose or multiple doses of a pharmaceutical composition of the invention, where contents from the vial can be drawn into the syringe for injection.

In some embodiments, the pharmaceutical composition may be administered to a patient by injection using a syringe system, including a syringe system as described herein. In aspects, the composition is injected by subcutaneous injection, although other parenteral routes, including intramuscular injection, are contemplated herein.

The composition may be administered by manual injection though a syringe with, for example, a 18 to 32 gauge needle, a 22 to 25 gauge needle, a 18 to 24 gauge needle, or an 18 to 22 gauge needle, or an 18 to 20 gauge needle, or may be administered by injection using an autoinjector.

Also contemplated herein is a kit comprising a prefilled syringe system disclosed herein.

The following experimental results are provided for purposes of illustration and are not intended to limit the scope of the invention.

### EXAMPLES

The following examples describes methods used to prepare and test the extended-release compositions comprising degarelix acetate (DgA) active pharmaceutical ingredient (API), or other forms of degarelix where the counter ions are formed with other acids such as poly-carboxylic acids (citric acid), strong acid (methane sulfonic acid), hydrophobic acid (pamoic acid, palmitic acid). The following examples discuss the preparation and use of only degarelix citrate form.

### Example 1

### 1. Preliminary Screening DgA-Drug Substance (DS) Solubility in Various Water Miscible Pharmaceutically Acceptable Organic Solvents:

Various organic solvents were screened for the maximal solubility of DgA-DS. Non-aqueous water-miscible pharmaceutically acceptable organic solvents like N-methyl-2-pyrrolidone (NMP), dimethyl sulfoxide (DMSO), and N, N-dimethylacetamide (DMA) have shown superior solubility capacity (at least 35-40% w/w) of degarelix than aqueous-based solvents. NMP and DMSO can dissolve higher amounts of DgA without inducing self-aggregation, as seen in aqueous solvents. Other solvents tested include-benzyl alcohol, PEG 300, propylene carbonate, triacetin, triethyl citrate, ethyl acetate, and benzyl benzoate. In the majority of these solvents, the drug is either insoluble or has solubility limits much lower than NMP, DMSO or DMA.

### 2. Degarelix Citrate (DgC)-DS Preparation

DgC-DS was formed by dissolving the degarelix as degarelix acetate (purchased from the vendor) in water (~40 mg/mL concentration) and then adding the aqueous solution of citric acid (IM) drop by drop. The molar ratio of degarelix free-base to citric acid in the solution was varied as desired. The resulting solution was mixed for 10-15 mins and placed in the −80° C. freezer. The contents were lyophilized to remove the water (and freeze-drying may remove some amount of acetic acid formed as a result of ion exchange). The dried powder was re-dissolved in water and freeze-dried again. The purity of the lyophilized degarelix citrate powder was tested using HPLC analysis. See descriptions of degarelix citrate drug substance powder in Table 1.

TABLE 1
Description of Degarelix Citrate (DgC)-Drug Substance powders

| Sample | Description |
|---|---|
| DgC-DS | The powder obtained after lyophilization of the aqueous |
| (1:2.4) | solution containing 1 mole of degarelix and 2.4 moles of the |
| | anhydrous citric acid |

### 3. Preparation of Dg-DS or Dg-DS/Organic-Solvent Formulations Bulk Solution and Prefilled Syringes

Bulk solution: To produce the drug/solvent bulk solution comprising the active pharmaceutical ingredient (DgA or DgC), the desired amount of the DgA-DS or DgC-DS was combined with the solvent NMP or DMSO or combination of solvents with or without co-solvent or other additives, in the indicated amounts (see individual experiments below). The API and solvent were combined in a glass vial or jar and blanketed with nitrogen. Jars were mixed using the jar mill, Turbula, or shaker at room temperature until homogeneous. DgA or DgC are very soluble in NMP or DMSO and can load higher concentrations (at least 40% w/w).

Syringe filling: After dissolving the API in the organic solvent, the liquid formulation was manually filled into syringes and capped with a tip cap. Syringes barrels selected for filling are made either of polypropylene material or cyclic olefin copolymer (COC) material. Filled syringes were then packaged in labeled foil pouches with a desiccant pack and the pouches were sealed. After filling the syringes with the formulations, the filled syringes were stored under refrigerated conditions (e.g., 2-8° C.) or accelerated conditions (≥25° C. see individual experiments). The syringes may be irradiated via e-beam or gamma irradiation (see individual experiment) for terminal sterilization.

### 4. In Situ Gelation Screening

Solutions of either DgA-DS in water (40 mg/mL), DgA-DS in NMP (~35% w/w), or DgA-DS in DMSO (~34% w/w) were injected into deionized water and phosphate buffer saline (PBS). When DgA solutions were injected into PBS (~50 mg of DgA injected into 10-15 mL), immediate aggregation and gelation of the peptide were observed, however, such a rapid aggregation and gelation effect was not observed when a similar amount of DgA solutions was injected into deionized water. The liquid in the vial post-injection became slightly turbid immediately and finally turned into a translucent gel overnight. NMP or DMSO-based formulation showed similar drug aggregation effects when injected into PBS as seen with aqueous-based DgA solution.

### 5. In Situ Gelation Vs Injection Volume

A solution of DgA-DS in DMSO (~35% w/w) was injected into 2.5 mL PBS (pH 7.4, 37° C.) at different injection volumes (10 μL, 25 μL, 50 μL, and 80 μL) to see if the aggregation/gelation behavior can be observed at smaller injection volumes. Irrespective of the injection volume, aggregation was seen at all levels. The extent of gelation (which can be observed by the thickness of the translucent gel phase) was higher for higher injection volume, which can be attributed to the higher drug amounts.

### 6. XRD Characterization of the DgA Solutions

Sample Preparation: The sample was loaded into a 2 mm by 20 mm zero-background sample holder using a spatula. The sample was made flush with the top of the sample holder using a microscope slide to press the sample down.

XRD Instrument Settings: The sample analysis was run using a Rigaku MiniFlex XRD instrument. The X-ray tube was set to 40 kV and 15 mA. The detector is D/tex Ultra 2, 2D detector. The filter is a KBNi (1.5). The scan mode was continuous with a scan axis of 2θ/θ a step width of 0.02 degrees and a scan speed of 5 degrees/minute. The scan range was 3°-45°. The arrangement of the optics was as follows: 5.0 degrees for the Incident Soller Slit, 0.625 mm for the Divergence Slit, 10.0 mm for the Incident Height Limiting Slit, 8.0 mm for the Soller Slit, 5.0 degrees for the Receiving Soller Slit, and open (no slit) for the Receiving Slit. The analysis was run at ambient temperature.

Analysis: The sample analysis was controlled using the MiniFlex software. After a sequence of samples was run, the data was processed using a separate software called PDXL2. Degarelix acetate being a biopolymer is amorphous not showing any crystallinity as an API powder or at a concentration of 66 mg/mL in water. At higher concentrations, the peptide forms a gel. The gel was not crystalline. In theory, the gel exhibits peaks indicating a higher order structure attributed to the stacking of beta-sheets forming aggregates resulting in the gel formation. The addition of salts can aid the gelation to form at a lower concentration than without the salts. No gelation occurs in the presence of NMP solvent even when water was present.

### 7. Analysis of Degarelix (Assay) and Total Related Compounds in the Formulations.

Transferred the formulation or dispensed the contents of the syringe into a 50 mL volumetric flask and recorded the weight. Diluted to volume with mobile phase A and mixed thoroughly by vortexing. Dilutions performed as needed-dilute 2 mL to 20 mL with mobile phase A (0.1% trifluoroacetic acid water) and mixed thoroughly by vortexing. Performed a second dilution of 2 mL to 20 mL with mobile phase A to obtain a working sample.

HPLC analysis: HPLC analysis for assay and related compounds is performed using an Agilent AdvanceBio Peptide 3.0×100 mm, 2.7 um column and an Agilent AdvanceBio Peptide Map Guard 3.0×5 mm 2.7 um guard column at 30° C. with a flow rate of 0.75 mL/min. The runtime was 15 minutes with a 10-μL injection for assay and related compounds for the solution gel depot formulation. The mobile phases were 0.1% trifluoroacetic acid water for mobile phase A and 0.1% trifluoroacetic acid acetonitrile for mobile phase B. The detection was UV and performed with a diode array detector set at 220 nm. The method used a gradient. See Table 2 below for the gradient method for an example chromatogram.

TABLE 2
Gradient Method for HPLC
HPLC Gradient Method

| | Mobile phase A | Mobile phase |
|---|---|---|
| Time | (%) | B (%) |
| 0.0 | 70 | 30 |
| 2.0 | 70 | 30 |
| 8.0 | 64 | 36 |
| 9.0 | 10 | 90 |
| 11.0 | 10 | 90 |
| 12.0 | 70 | 30 |
| 15.0 | 70 | 30 |

8. Recovery of Drug from Different Degarelix/Organic Solvent Formulations

Prefilled syringes of degarelix/organic solvent formulations were prepared as described in Section 3. Recovery of drugs from different formulations was tested using the HPLC method (see Section 7) and is listed in Table 3.

Table 3 Observations: All the formulations showed good recoveries (90.0-110.0%), indicating the applicability of the method and the initial stability of the API in different solvent systems.

TABLE 3
Drug recovery testing from prefilled syringes of organic-solvent
based formulations

| | | | | | Average | |
|---|---|---|---|---|---|---|
| | | | | | (±SD | |
| | | | | Average | Recovery | |
| | | | | (±SD) | of | |
| | | | | degarelix | Degarelix | |
| | | Degarelix | Degarelix | conc. | from | Total |
| | | conc. in | conc. in | in | Prefilled | Related |
| | | bulk | Prefilled | Prefilled | Syringe | Com- |
| | | solution | Syringe | Syringe | (% w/w | pounds |
| F# | Composition | (% w/w) | (% w/w) | (% w/w) | assay) | (%) |
| F1 | DgA- | 34.6 | 34.6 | 35.0 ± 0.4 | 101.0 ± 1.2 | 0.00 |
| | DS/NMP | | 34.8 | | | |
| | 35/65 | | 35.4 | | | |
| F2 | DgA- | 34.2 | 35.1 | 35.1 ± 0.3 | 102.7 ± 0.8 | 0.00 |
| | DSDMSO | | 34.9 | | | |
| | 35/65 | | 35.4 | | | |
| F3 | DgA- | 34.2 | 35.4 | 35.0 ± 0.8 | 102.2 ± 1.9 | 0.00 |
| | DS/NMP/ | | 35.2 | | | |
| | BnOH | | 34.2 | | | |
| | 35/60/5 | | | | | |
| F4 | DgC- | 27.2 | 29.7 | 28.6 ± 1.0 | 105.2 ± 3.6 | 0.00 |
| | DS/NMP | | | | | |
| | 35/65 | | | | | |

(“F#” denotes “Formulation Number”)

### 9. Stability of Degarelix/Organic Solvent Formulations

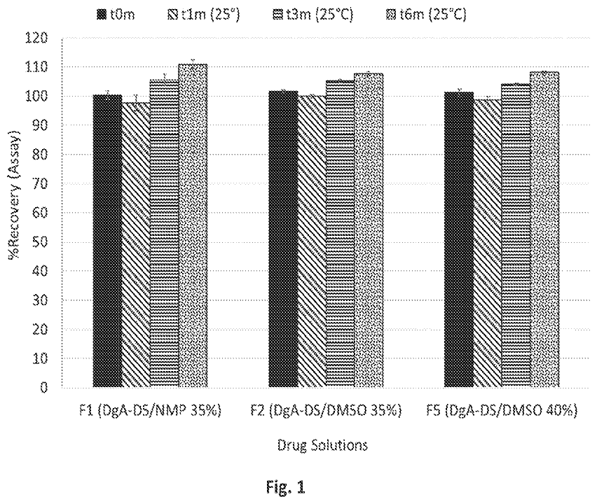

Prefilled syringes of degarelix/organic solvent formulations were prepared as described in Section 3. The samples were not e-beamed. The composition of the samples is shown in Table 4. The sealed foil pouches containing prefilled syringes were placed at accelerated storage conditions (25±2° C. for 6 months) and tested for drug assay or recovery and related compounds (see section 7 for procedure) at each time pull (0, 1, 3, and 6 months). The results of this study are presented in FIG. 1. No degradation of the drug was observed at 6 months, and a slight increase in percentage recoveries over time could be attributed to the solvent loss/absorption in prefilled syringes at accelerated conditions.

TABLE 4
Composition of formulations

| F# | Composition (Description) | Sample type |
|---|---|---|
| F1 | Degarelix Acetate Drug | Prefilled syringes |
| | Substance (DgA-DS)/NMP | (polypropylene barrel, non-e- |
| | 35/65% w/w | beamed); Syringes were foil |
| F2 | DgA-DS/DMSO 35/65% w/w | pouched with a desiccant bag |
| F5 | DgA-DS/DMSO 40/60% w/w | |

(“F#” denotes “Formulation Number”)

FIG. 1 Observations: Degarelix acetate was found to be stable in NMP and DMSO (at studied concentrations) at 25° C. for 6 months (accelerated conditions). No significant increase in the related compounds. At 6 months, the total related compounds were below 2%. The observed slight increase in percentage recoveries for the F1, F2, and F5 formulations on storage could be attributed to the solvent loss over time at accelerated storage conditions from the prefilled syringes or stopper absorption. No gelation was observed in the formulations F1, F2, and F5. F5 formulation was made at a higher concentration than F2, and both were found to be stable and did not show any gelation. This supports that higher concentration such as 40% w/w is feasible too.

10. Terminal Sterilization Feasibility Via e-Beam or Gamma Irradiation

The prefilled syringes of degarelix/organic solvent formulations were exposed to e-beam or gamma radiation at different doses and tested the drug concentrations or recoveries for pre and post-irradiation samples using HPLC (see section 7) and the results are presented in Table 5.

TABLE 5
Results of irradiation feasibility studies

| | | | | | | Degarelix | |
|---|---|---|---|---|---|---|---|
| | | Degarelix | | | | Conc. in | |
| | | Conc. in | | Dose | Dosimeter | post-Irr. | |
| | Composition | pre-Irr. sample | Irradiation | Delivered | Reading | sample | % Change |
| F# | (% w/w) | (% w/w) | Type | (kGy) | (kGy) | (% w/w) | in Conc. |
| F6 | DgA-DS powder | 86.4 | e-beam | 25 | 21.5 | 87.6 | 1.4 |
| F1- | 35/65 DgA- | 31.4 | e-beam | 25 | 21.5 | 30.9 | −1.8 |
| e25- | DS/NMP | | | | | | |
| 1 | | | | | | | |
| F5- | 40/60 DgA- | 36.2 | e-beam | 25 | 21.5 | 35 | −3.5 |
| e25 | DS/DMSO | | | | | | |
| F1- | 35/65 DgA- | 33.7 | e-beam | 34 | 29.1 | 33.1 | −1.9 |
| e34 | DS/NMP | | | | | | |
| F6- | 40/60 DgA- | 40 | e-beam | 34 | 29.1 | 37 | −7.4 |
| e34 | DS/NMP | | | | | | |
| F2- | 35/65 DgA- | 32.7 | e-beam | 34 | 29.1 | 33 | 0.9 |
| e34 | DS/DMSO | | | | | | |
| F5- | 40/60 DgA- | 37.1 | e-beam | 34 | 29.1 | 34.5 | −7 |
| e34 | DS/DMSO | | | | | | |
| F3- | 35/60/5 DgA- | 32.6 | e-beam | 34 | 29.1 | 31.9 | −2.1 |
| e34 | DS/NMP/BnOH | | | | | | |
| F7- | 35/55/10 DgA- | 33.4 | e-beam | 34 | 29.1 | 32 | −4.1 |
| e34 | DS/NMP/BnOH | | | | | | |
| F1- | 35/65 DgA- | 33.7 | gamma | 18-27 | 19.9-26.4 | 32.4 | −3.7 |
| g22 | DS/NMP | | | | | | |
| F6- | 40/60 DgA- | 40 | gamma | 18-27 | 19.9-26.4 | 37.7 | −5.7 |
| g22 | DS/NMP | | | | | | |
| F2- | 35/65 DgA- | 32.7 | gamma | 18-27 | 19.9-26.4 | 32.2 | −1.4 |
| g22 | DS/DMSO | | | | | | |
| F5- | 40/60 DgA- | 37.1 | gamma | 18-27 | 19.9-26.4 | 37.9 | 2.2 |
| g22 | DS/DMSO | | | | | | |
| F3- | 35/60/5 DgA- | 32.6 | gamma | 18-27 | 19.9-26.4 | 30.5 | −6.6 |
| g22 | DS/NMP/BnOH | | | | | | |
| F7- | 35/55/10 DgA- | 33.4 | gamma | 18-27 | 19.9-26.4 | 32.9 | −1.4 |
| g22 | DS/NMP/BnOH | | | | | | |
| F1- | 35/65 DgA- | 35 | e-beam | 25 | 21.8 | 34 | −2.9 |
| e25- | DS/NMP | | | | | | |
| 2 | | | | | | | |
| F2- | 35/65 DgA- | 35.1 | e-beam | 25 | 21.8 | 33.7 | −4.1 |
| e25- | DS/DMSO | | | | | | |
| 2 | | | | | | | |
| F3- | 35/60/5 DgA- | 35 | e-beam | 25 | 21.8 | 34.3 | −2 |
| e25- | DS/NMP/BnOH | | | | | | |
| 2 | | | | | | | |
| F4- | 35/65 DgC- | 28.6 | e-beam | 25 | 21.8 | 28.5 | −0.3 |
| e25 | DS/NMP | | | | | | |
| 2 | | | | | | | |

(“F#” denotes “Formulation Number”)

Table 5 Observations: These results showed that depending on the irradiation type, irradiation dose, concentration of the drug, salt form, and stabilizer, the degarelix solutions in an organic solvent are feasible for terminal sterilization by irradiation.

F4-e25-2 formulation degarelix citrate showed better stability upon e-beam irradiation compared to degarelix acetate formulation F1-e25-2.

In general, 35% of drug solution samples (F1-e34, F2-e34, F1-g22) showed better stability upon irradiation compared to 40% of drug solution formulations (F6-e34, 5-e34, F6-g22).

Formulations F1-e25, F2-e25, F3-e25, and F4-e25 were tested for total related compounds, no significant increase was observed post-irradiation of the samples via e-beam.

### 11. Stability of Irradiated Samples in Accelerated Storage Conditions

Irradiated prefilled syringes of degarelix/organic solvent formulations were stored at different accelerated storage conditions (25° C.) and tested for assay at 30 and 120 days using the HPLC method (see section 7). Results are presented in Table 6.

TABLE 6
Accelerated storage stability testing of irradiated prefilled syringes of degarelix
solution formulations

| | | Post-e-beam (29.1 kGy) | | | | Post-gamma (19.9-26.4 | | |
|---|---|---|---|---|---|---|---|---|
| | | storage time at 25° C. | | | | kGy) storage time at 25° C. | | |
| Sample/Formulation | | T = 0 m | T = 30 d | T = 120 d | | T = 0 m | T = 30 d | T = 120 d |
| Description | F# | Drug Conc. (% w/w) | | | F# | Drug Conc. (% w/w) | | |
| 35/65 Degarelix | F1-e34 | 33.1 | 31.6 | 34.2 | F1-g22 | 32.4 | 32.0 | 35.3 |
| Acetate/NMP | | | | | | | | |
| 40/60 Degarelix | F6-e34 | 37.0 | 36.4 | NA | F6-g22 | 37.7 | 34.8 | NA |
| Acetate/ NMP | | | | | | | | |
| 35/65 Degarelix | F2-e34 | 33.0 | 34.6 | 34.9 | F2-g22 | 32.2 | 32.5 | 36.1 |
| Acetate/DMSO | | | | | | | | |
| 40/60 Degarelix | F5-e34 | 34.5 | 35.2 | NA | F5-g22 | 37.9 | 33.4 | NA |
| Acetate/DMSO | | | | | | | | |
| 35/60/5 Degarelix | F3-e34 | 31.9 | 31.4 | 35.8 | F3-g22 | 30.5 | 31.9 | 38.5 |
| Acetate/NMP/BA | | | | | | | | |
| 35/55/10 Degarelix | F7-e34 | 32.0 | 31.1 | 35.6 | F7-g22 | 32.9 | 33.8 | 35.4 |
| Acetate/NMP/BA | | | | | | | | |

(“F#” denotes “Formulation Number”)
“NA” indicates: Not Available

Table 6 Observations: No pronounced degradation of the degarelix was observed in the irradiated samples over the studied period and a slight increase in percentage recoveries over time could be attributed to the solvent loss or absorption in prefilled syringes at accelerated storage conditions.

### 12. Viscosity of the Formulation

500 μL of the formulation was transferred into a Hamilton syringe and the viscosity of the formulation was measured using RheoSense. Before measurement, an equilibration step was performed on the instrument to determine the best instrument parameters before running the test. Results are presented in Table 7. Viscosities of the formulations were in the range of 100-250 cP.

TABLE 7
Viscosity of the formulations post-e-beam
irradiation of the prefilled syringes

| F# | Composition | Avg. Viscosity (cP) |
|---|---|---|
| F1-e25 | DgA-DS/NMP 35/65 | 185 ± 4.3 |
| F2-e25 | DgA-DSDMSO 35/65 | 129 ± 1.0 |
| F3-e25 | DgA-DS/NMP/BnOH 35/60/5 | 217 ± 2.8 |
| F4-e25 | DgC-DS/NMP 35/65 | 154 ± 0.8 |

(“F#” denotes “Formulation Number”)

### 13. Nonclinical Evaluation of the Formulations for Drug Release

Prefilled syringes of degarelix/organic solvent formulations were prepared as described in Section 3. The samples were not e-beamed. Degarelix release rates of these formulations were obtained using a rat model. Male rats were each injected with a single subcutaneous injection of the degarelix/organic solvent formulations. The composition of the formulations and the dosing details are listed in Table 8. Formulation F1 examined the effect of organic solvent, as compared to control (reconstituted in water); Formulation F2 examined the effect of the type of organic solvent in the formulation (NMP vs. DMSO); Formulation F3 examined the effect of adding benzyl alcohol to the formulation; Formulation F4 examined the effect of a different salt form of degarelix (citrate salt) on the formulation.

At predetermined time points, rats were bled and plasma degarelix levels were determined using liquid chromatography with tandem mass spectrometry (LC-MS/MS). Each data point is based on an average plasma degarelix concentration. Six rats were dosed for each group, with sparse blood sampling performed for early time points. FIRMAGON® (degarelix for injection) was included in the study as a control. The control was injected on day 0 and Day 28 at 15 mg/kg to understand steady state and accumulation, whereas test groups received a single injection of 15 mg/kg. Dosing was calculated using the amount of degarelix free base in each formulation which was calculated for the formulation after correction for purity. A 120 mg vial of FIRMAGON® was reconstituted by adding 3 mL of sterile water for injection provided with the FIRMAGON® kit, and the resulting reconstituted solution was at 40 mg/mL concentration of degarelix.

TABLE 8
Non-Clinical Study Dosing Details

| Formulation | | Nominal | Degarelix | Target Dose | Wt. of | Volume |
|---|---|---|---|---|---|---|
| # | | Composition | Acetate | in Rat | formulation | injected |
| (Group ID) | Description | (% w/w) | Conc. (% w/w) | Model | injected (mg) | (mL) |
| Control | Control | Degarelix | 4% | 15 mg/kg | 114.1 ± 5.2 | 115 μL |
| (A-CA1) | (FIRMAGON | Acetate in | | (4.5 mg/rat | | |
| | 120 mg) | sterile WFI | | of 0.3 kg | | |
| | | (40 mg/mL) | | size) | | |
| F1 | Test Articles | DgA-DS/NMP | 35.0 | | 14.9 ± 0.6 | 14 μL |
| (B-TA1) | (Degarelix/ | 35/65 % w/w | | | | |
| F2 | Organic | DgA-DS/ | 35.1 | | 12.0 ± 3.4 | 14 μL |
| (C-TA2) | Solvent) | DMSO 35/65 | | | | |
| | solution | % w/w | | | | |
| F3 | | DgA- | 35.4 | | 13.2 ± 3.4 | 14 μL |
| (D-TA3) | | DS/NMP/BnO | | | | |
| | | H 35/60/5 | | | | |
| | | % w/w | | | | |
| F4 | | DgC-DS/NMP | 28.6 | | 14.3 ± 2.2 | 14 μL |
| (E-TA4) | | 35/65 % w/w | | | | |

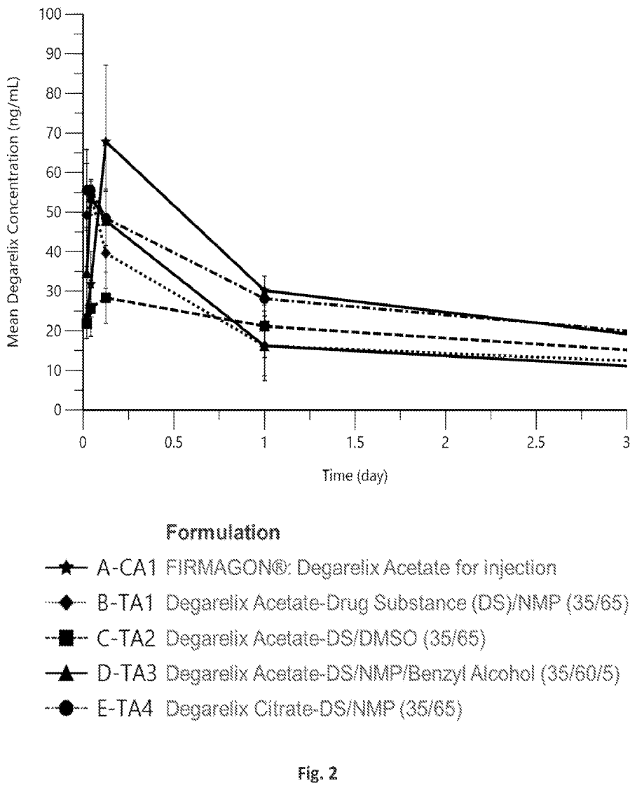

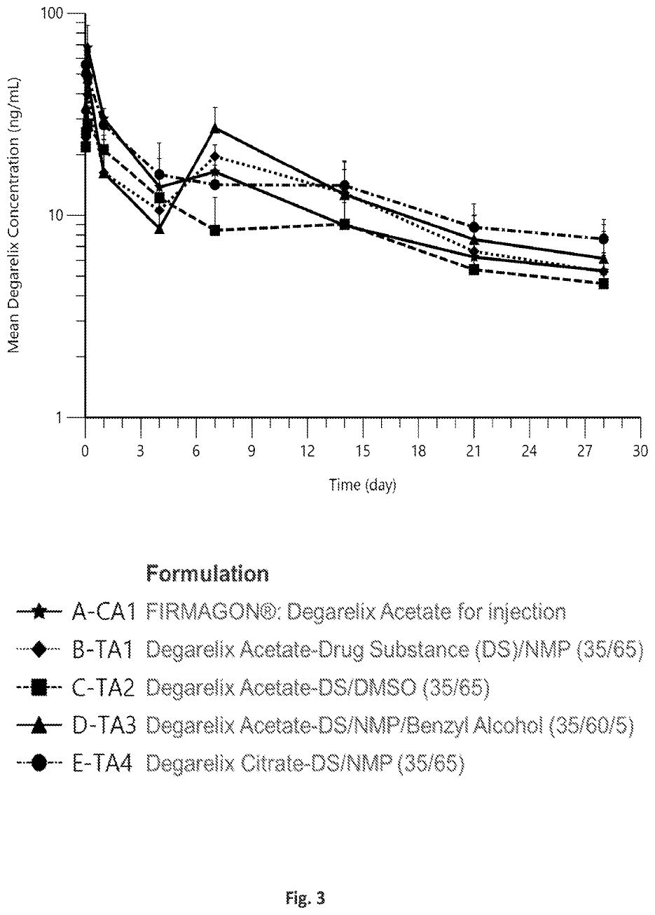

In vivo degarelix release in rats for organic solvent-based formulations of degarelix formulations at 28-days post-injection is presented in FIG. 2 and FIG. 3. FIG. 2 is a magnified version of Day-0 to Day-3 levels to show the Cₘₐₓ levels.

FIG. 2 and FIG. 3 Observations: All the organic solvent-based degarelix formulations (Groups B-TA1, C-TA2, D-TA3, and E-TA4) exhibited sustained release profiles of the drug, and the drug was detected for at least 28 days.

All the organic solvent-based degarelix formulations (Groups B-TA1, C-TA2, D-TA3, and E-TA4) exhibited lower initial release (Cmax) than control (Group A-CA1) which could be attributed to the i) solvent used in the formulation (water in the control vs. organic solvent in the test formulations) and ii) difference in the gelation kinetics of the drug from different formulations.

There are three levels of Cmax observed for these set of test articles:
Higher Cmax—for water based control formulation A-CA1
Medium Cmax—for NMP based formulations B-TA1, D-TA3, and E-TA4
Lower Cmax—for DMSO based formulation for C-TA2

These results suggest the primary/major solvent of the formulation has higher impact on the initial release (Cmax) of the drug than the salt form or co-solvent.

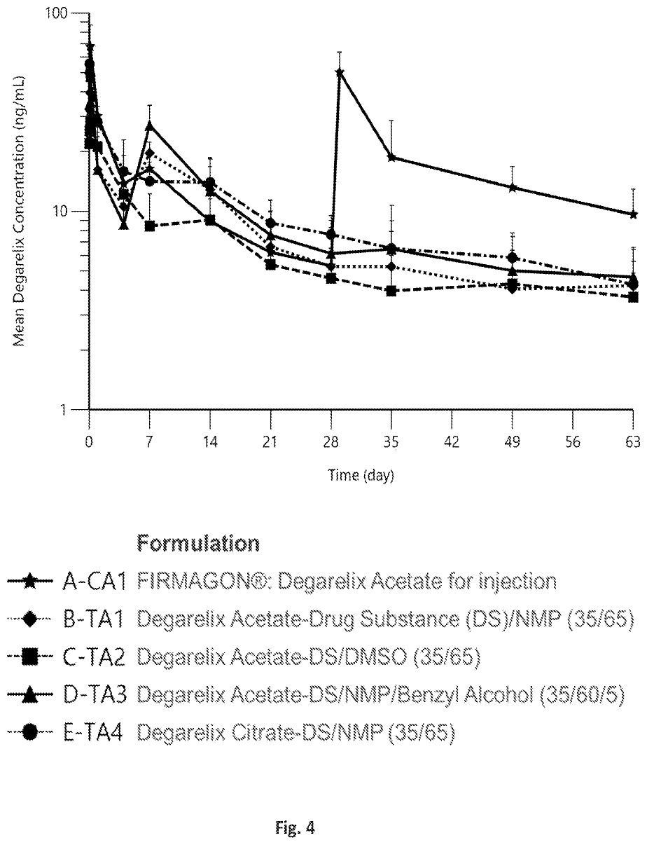

In vivo degarelix release in rats for the solvent-based formulations of degarelix at 63-days post injection is presented in FIG. 4. FIG. 4 shows that all of the formulations released drug that resulted in measurable levels of degarelix in vivo for greater than 60 days.

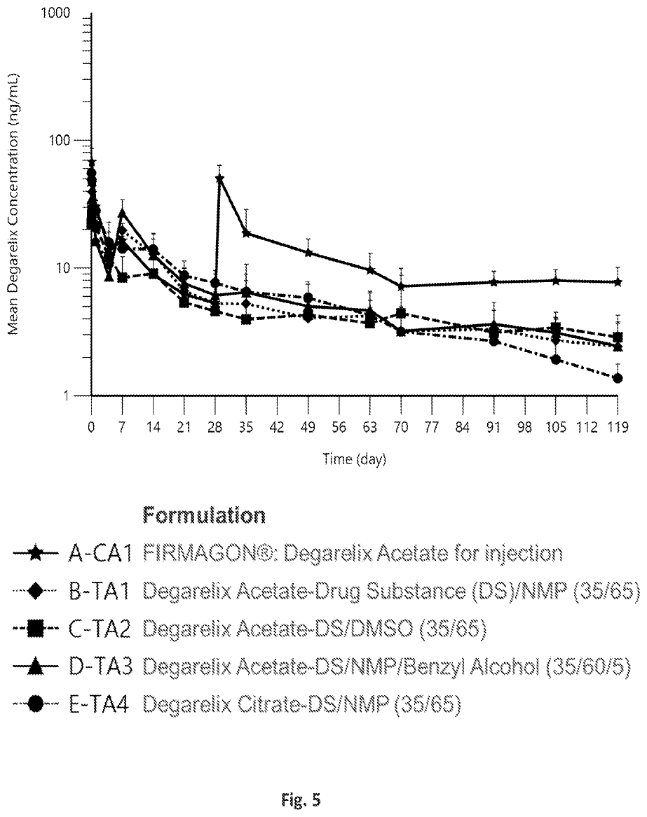

In vivo degarelix release in rats for solvent-based formulations of degarelix at 119-days post injection is presented in FIG. 5. FIG. 5 shows that all of the formulations released drug that resulted in measurable levels of degarelix in vivo for greater than 90 days (at least 119 days).

Table 9 shows the PK data for the experiment described above. The Cmax for formulations B-TA1, D-TA3, and E-TA4 occurs earlier for solvent-based formulations compared to Control (A-CA-1); this could lead to a faster reduction in testosterone levels.

TABLE 9
pK Data.

| | | AUC0-1 | AUClast | AUClast_D | | | Cmax_D |
|---|---|---|---|---|---|---|---|
| | Dose | (day*ng/ | (day*ng/ | (day*kg*n | Tmax | Cmax | (kg*ng/ |
| Group | (mg/kg) | mL) | mL) | g/mL/mg) | (day) | (ng/mL) | mL/mg) |
| A-CA1 | 14.21/13.14* | 47.86 | 1394.16 | 50.97 | 0.13 | 67.8 | 2.48 |
| B-TA1 | 16.21 | 29.97 | 671.91 | 41.45 | 0.04 | 54.33 | 3.35 |
| C-TA2 | 13.14 | 24.66 | 593.67 | 45.18 | 0.13 | 28.37 | 2.16 |
| D-TA3 | 14.37 | 33.57 | 759.93 | 52.88 | 0.04 | 53.77 | 3.74 |
| E-TA4 | 12.75 | 39.6 | 735.75 | 57.71 | 0.02 | 55.53 | 4.36 |

*Dosed once on Day 0 and once on Day 28. Dose normalized parameters are calculated off total dose

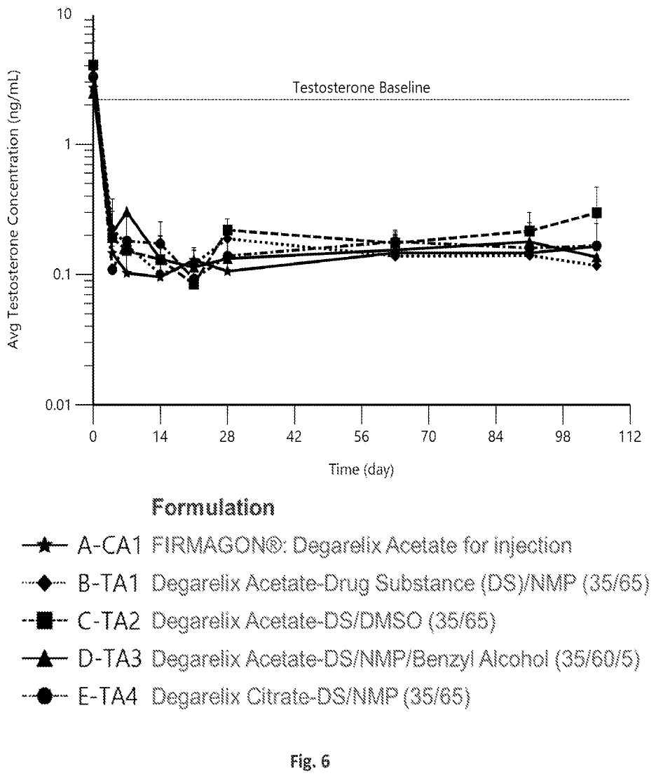

In vivo testosterone concentration levels (ng/mL) in rats for the solvent-based formulations of degarelix in this example at 105-days post injection is presented in FIG. 6. FIG. 5 shows that all of the formulations lasted the duration of the 119 day study. Further, the results show that all of the formulations had plasma concentration of greater than 1 ng/mL which is the minimum drug concentration to induce chemical castration. In addition, the testosterone level remained below the “historical testosterone baseline” level and that testosterone suppression occurred by day 4. Lastly, a second dose of FIRMAGON® (A-CA1) at Day 28 did not suppress testosterone concentration levels any more than the other formulations dosed once on Day 0.

### Example 2

This example describes the results of a non-clinical animal study demonstrating the effects of the addition of an additive in certain extended-release degarelix formulations on in vivo pharmacokinetics (PK) and testosterone levels (pharmacodynamics, PD) in male Sprague Dawley rats after subcutaneous injection of the formulations.

This study includes a control formulation (such as FIRMAGON®), and the degarelix formulations as listed in Table 10 below. The additives included in the degarelix formulations, if present, were either acetic acid (AcOH) or benzyl alcohol (BnOH). At the conclusion of the study, rats were euthanized, and injection sites were collected for histological and residual drug analysis. After these degarelix formulations and control formulation were delivered into male rats via subcutaneous injection, the rats were periodically bled so that plasma degarelix and testosterone levels are assessed.

TABLE 10

| Formulation Group and | | | Solvent | Additive | Terminal |
|---|---|---|---|---|---|
| Composition (% w/w) | | API (wt %)¹ | (wt %) | (wt %) | Sterilization |
| B: | 35/62.8/2.2 | 35% Dg Acetate | 62.8% | 2.2% | E-beam |
| | DgA/NMP/AcOH | | NMP | AcOH | |
| E: | 24/56/20 DgA/NMP/BnOH | 24% Dg Acetate | 56% | 20% BnOH | E-beam |
| | | | NMP | | |
| H: | 24/73.3/1.7 | 24% Dg Acetate | 73.3% | 1.7% | E-beam |
| | DgA/NMP/AcOH | | NMP | AcOH | |
| I: | 35/65 DgA/DMSO | 35% Dg Acetate | 65% | N/A | E-beam |
| | | | DMSO | | |
| J: | 35/62.8/2.2 | 35% Dg Acetate | 62.8% | 2.2% | N/A |
| | DgA/NMP/AcOH | | NMP | AcOH | |
| | (no e-beam) | | | | |

¹API percentage refers to Degarelix acetate, not peptide (degarelix free base) content.
DgA = degarelix acetate drug substance; AcOH = Acetic acid; BnOH = Benzyl alcohol; NMP = N-methyl-2-pyrrolidone; DMSO = dimethyl sulfoxide.

### Degarelix Bulk Solution Preparation and Syringe Fill

Drug substance (DgA), and solvent with or without additive as indicated (NMP: acetic acid, or NMP: benzyl alcohol, or DMSO) were combined in a 40 mL amber colored scintillation vial and blanketed with nitrogen. The vials were mixed at room temperature until completely dissolved as indicated by a clear solution with no visible solids. Bulk drug solution was filled into labeled male syringes manually and capped with a female tip cap. Syringes were packaged in labeled foil pouches with a desiccant pack and the pouches were sealed. A portion of the syringes was sent for e-beam irradiation for stability testing as indicated in Table 10. Samples were irradiated at a targeted delivered dose range of 34-34 kGy, and the internal dosimeter reading on the syringe was 31.7 kGy. Samples were refrigerated at 5° C. until use.

### Non-Clinical Animal Study Dosing

Prefilled syringes of degarelix test formulations were prepared as described above. Degarelix release rates of these formulations as well as resulting testosterone levels were obtained using a rat model. Male rats were each injected with a single subcutaneous injection of the degarelix test and control formulations. The composition of the formulations and the dosing details are listed in Table 11. At predetermined time points, rats were bled and plasma degarelix levels were determined using liquid chromatography with tandem mass spectrometry (LC-MS/MS). Testosterone levels were also determined. Each data point is based on an average plasma degarelix concentration. Six rats were dosed for each group, with sparse blood sampling performed for early time points (Days 0 through Day 7). FIRMAGON® (degarelix for injection, Ferring) was included in the study as a control. The control was injected on day 0 and Day 28 at 45 mg/kg to understand steady state and accumulation, whereas test groups received a single injection of 45 mg/kg. Dosing for the test formulations was calculated using the amount of degarelix acetate in each formulation (at this dose volume there is not a significant difference in the amount of formulation given if dosing was alternatively calculated based on degarelix free base in the formulation after correction for purity). One vial of FIRMAGON® 240 mg kit contains 2 vials of 120 mg of degarelix. Each vial is reconstituted with 2 mL water from the prefilled syringe containing 3 mL of Sterile Water for Injection (provided in the kit). The final API is at a concentration of 60 mg/mL.

TABLE 11
Non-Clinical Study Dosing Details

| | Formulation Group | | | | |
|---|---|---|---|---|---|
| | and Composition | Degarelix | Target Dose of | Actual volume | Actual weight of |
| Description | (% w/w)¹ | (% w/w)² | Degarelix (mg) | dosed (μL) | formulation (mg) |
| Control | A: Firmagon | 6% w/v | 45 mg/kg | 225 | 233.9 ± 0.82 |
| | | (60 mg/mL) | (13.5 mg/rat of | | |
| Degarelix | B: 35/62.8/2.2 | 32.39 | 0.3 kg size) | 60 | 38.1 ± 6.37 |
| Test | DgA/NMP/AcOH | | | | |
| Formulations | E: 24/56/20 | 21.92 | | 70 | 57.7 ± 5.48 |
| | DgA/NMP/BnOH | | | | |
| | H: 24/73.3/1.7 | 21.84 | | 70 | 60.58 ± 0.97 |
| | DgA/NMP/AcOH | | | | |
| | I: 35/65 DgA/DMSO | 31.67 | | 60 | 37.9 ± 9.3 |
| | J: 35/62.8/2.2 | 32.18 | | 60 | 42 ± 7.39 |
| | DgA/NMP/AcOH | | | | |
| | (no e-beam) | | | | |

¹Target API is 35% or 24% wt % degarelix acetate (DgA) drug substance, as indicated for each formulation.
NMP = N-methyl-2-pyrrolidone; DMSO = dimethyl sulfoxide; AcOH = acetic acid; BnOH = benzyl alcohol.
²The actual amount of degarelix as degarelix free base in the formulation (degarelix free base equivalent), after correction for purity.

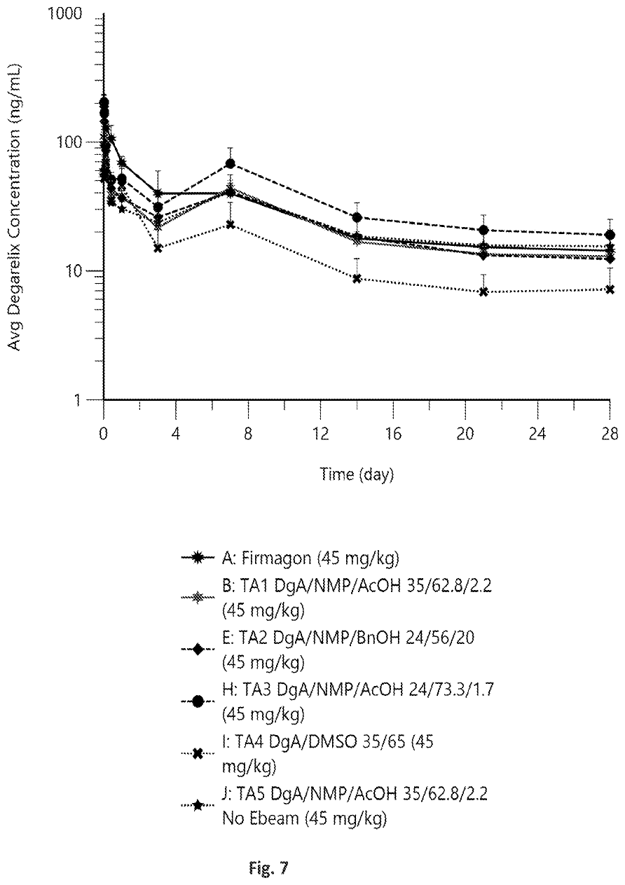

In vivo degarelix release in rats for organic solvent-based degarelix formulations at 28-days post-injection is presented in FIG. 7.

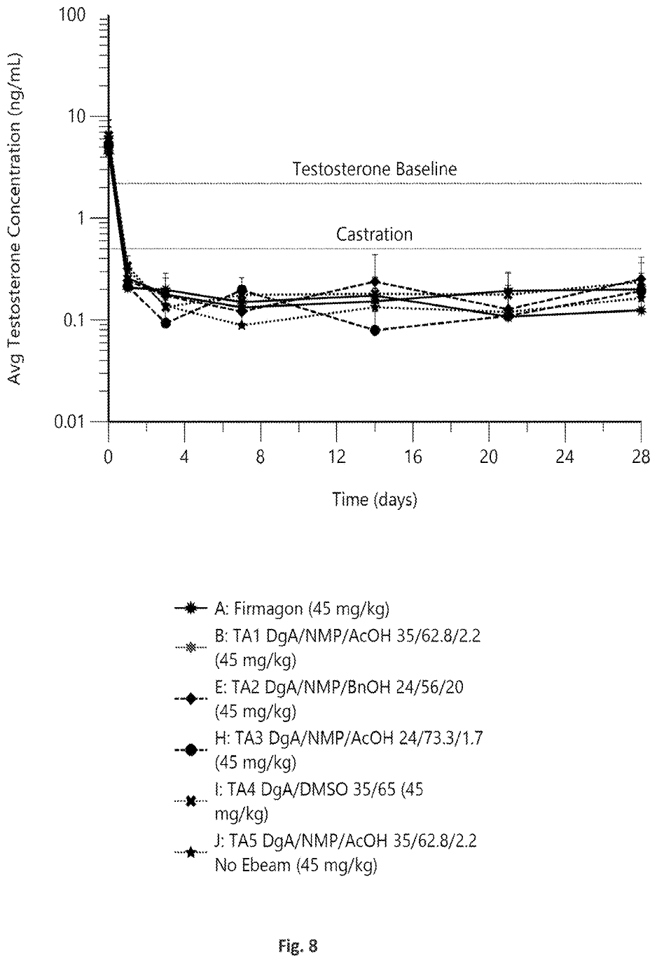

In vivo testosterone levels in rats for organic solvent-based degarelix formulations at 28-days post-injection is presented in FIG. 8.

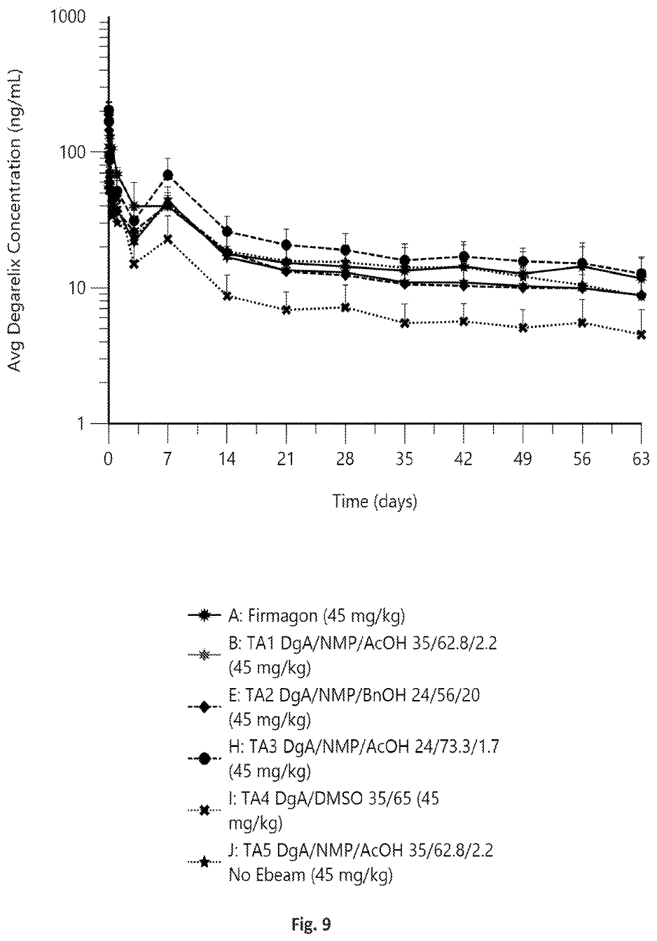

In vivo degarelix release in rats for the solvent-based degarelix formulations at 63-days post injection is presented in FIG. 9.

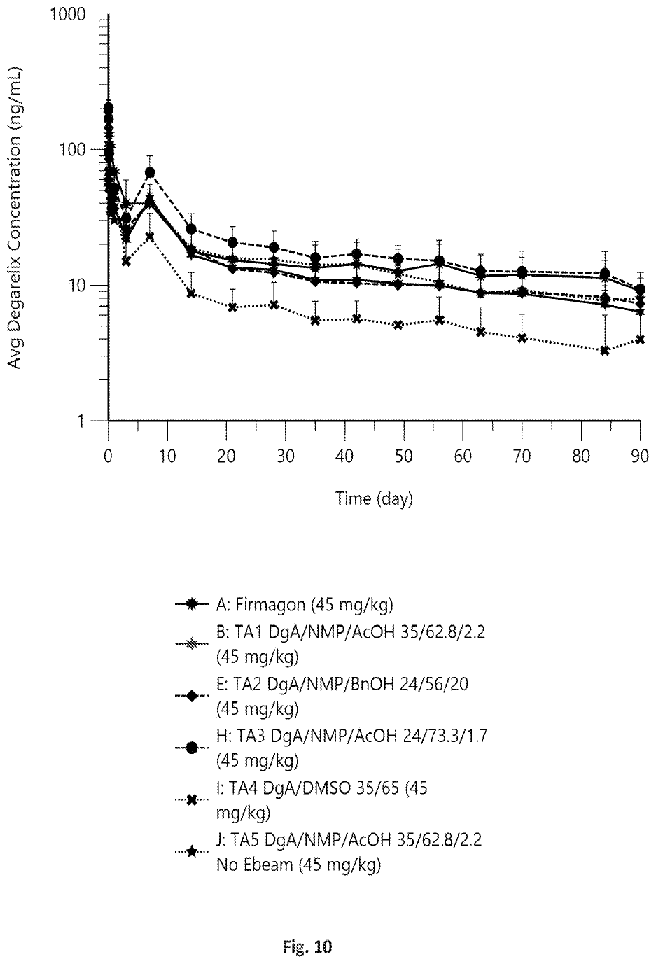

In vivo degarelix release in rats for solvent-based degarelix formulations at 90-days post injection is presented in FIG. 10.

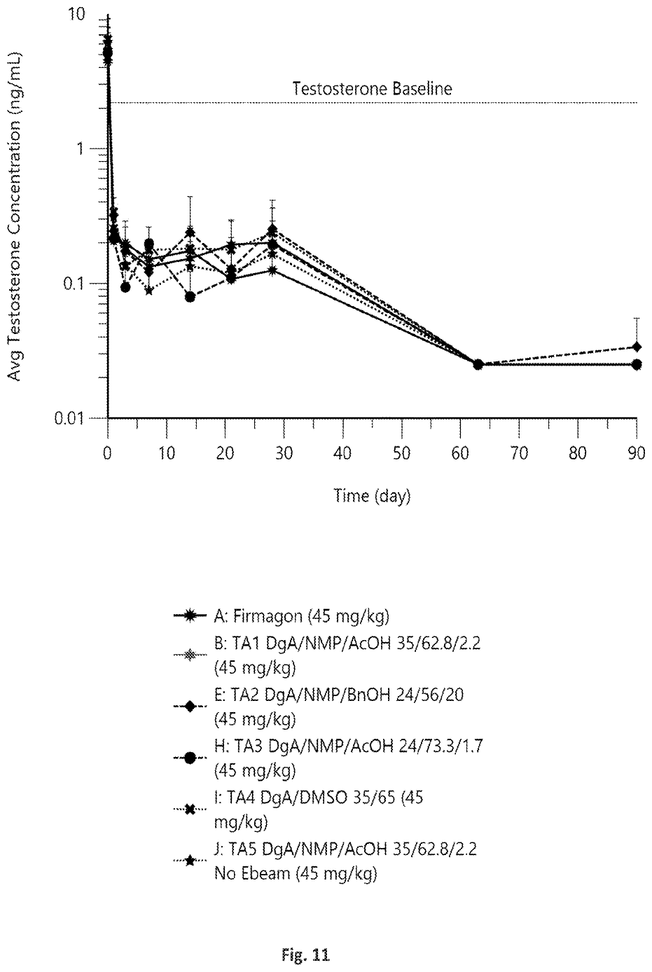

In vivo testosterone levels in rats for organic solvent-based degarelix formulations at 90-days post-injection is presented in FIG. 11.

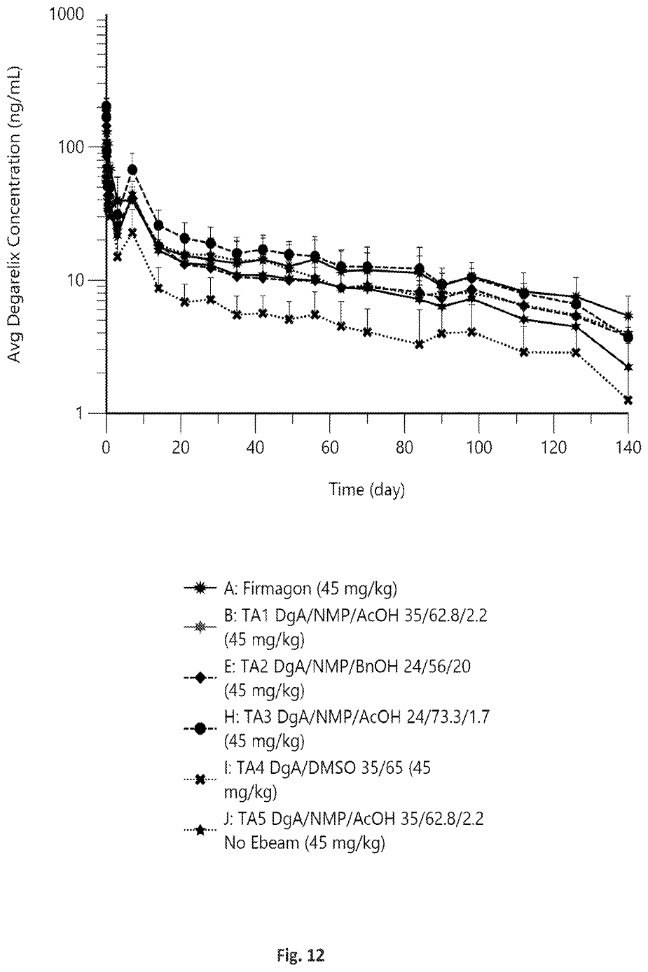

In vivo degarelix release in rats for solvent-based degarelix formulations at 140-days post injection is presented in FIG. 12.

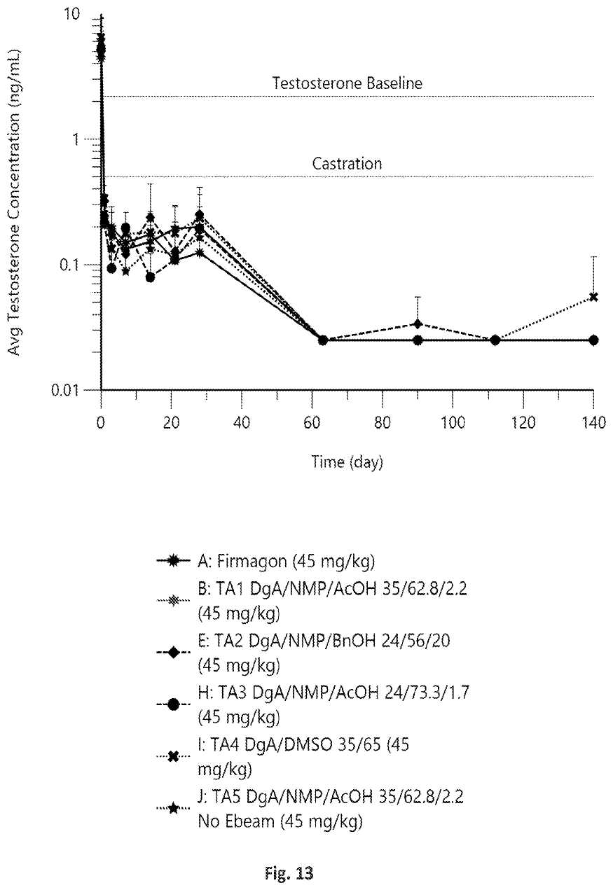

In vivo testosterone levels in rats for organic solvent-based degarelix formulations at 140-days post-injection is presented in FIG. 13.

The results presented in this example demonstrate that all of the degarelix test formulations tested herein maintained the minimum degarelix concentration of 1 ng/ml required for castration through greater than 4 months. In addition, testosterone suppression to below castration concentrations occurs by day 1 in all of the degarelix test formulations tested herein, as well as the control dosed at 45 mg/kg (note control was dosed at both Days 0 and 28, whereas test formulations were dosed as a single dose on Day 0). Further, testosterone suppression does not appear to be dose-dependent (data not shown). Additionally, clinical observations do not seem to impact drug release or testosterone suppression, as the control, formulation B (DgA/NMP/AcOH), formulation I (Dg/DMSO), and formulation J (Dg/NMP/AcOH-no ebeam) at 45 mg/kg all had either no observations or minimal observations. Gelation through the first 7 days appears to be different compared to the control, which, without being bound by theory, may be due to the solvents in the test formulation and resulting gelation strength.

### Example 3

This example demonstrates the effects of acid additive content on degarelix formulation stability. This study was designed to determine the degarelix formulation stability when varying levels of different acids as excipients are added to degarelix formulations of the invention.

A pH study was initiated to study the effects of pH control as a model of stability of degarelix formulations using a control without acid additive, or with the addition of an acid additive of acetic acid, citric acid, or succinic acid in three ratios and placed on stability to monitor impurity growth. A control sample of degarelix acetate and NMP (DgA/NMP 35%/65% w/w), which did not contain acid additive as excipient, was compared to other formulations of the same composition that were spiked with acid excipients at various molar ratios of degarelix peptide (calculated as free base) to acid (acetic acid, citric acid, or succinic acid) (see Table 12). Samples were placed under 5° C., 25° C., and 40° C. conditions and tested for a duration of 3-12 months. Testing included assay recoveries, related compounds and pH analysis.

TABLE 12

| | | Molar Ratio |
|---|---|---|
| Group | Formulation Description | Peptide to Acid Excipients |
| No acid | DgA-DA/NMP 35/65 | No Acid Excipient |
| (control) | | |
| Acetic acid | DgA-DA/NMP 35/65 + Acetic Acid | 1:1 |
| | DgA-DA/NMP 35/65 + Acetic Acid | 1:3 |
| | DgA-DA/NMP 35/65 + Acetic Acid | 1:5 |
| Citric acid | DgA-DA/NMP 35/65 + Citric Acid | 10:1 |
| | DgA-DA/NMP 35/65 + Citric Acid | 1:1 |
| | DgA-DA/NMP 35/65 + Citric Acid | 1:2 |
| Succinic acid | DgA-DA/NMP 35/65 + Succinic Acid | 1:1 |
| | DgA-DA/NMP 35/65 + Succinic Acid | 1:2 |
| | DgA-DA/NMP 35/65 + Succinic Acid | 1:3 |

DgA-DA = degarelix acetate; NMP = N-methyl-2-pyrrolidone.

### CONCLUSIONS

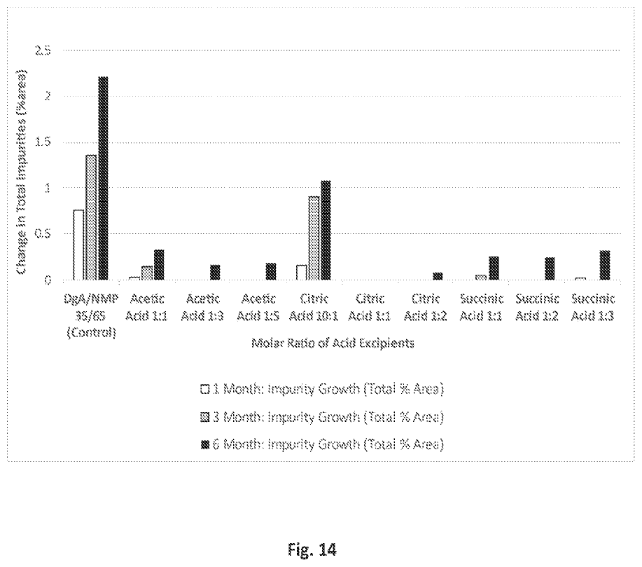

NMP has a pH of approximately 8-9 (National Center for Biotechnology Information, 2023) and, in the presence of water, the pH of the formulations decreases as more H+ ions flow freely. Consideration of how subcutaneous environments affect the pH of these formulations may be beneficial. Compared to the control sample without acid excipient, in this experiment, all formulations with acid excipients at all ratios showed better stability at both the 25° C. and the 40° C. accelerated conditions at 6- and 3-months, respectively. All three acids in this study reduced the formulation apparent pH to a new value that is dependent on both the amount of acid added in and its pKa. The impurity growth shown at the 40° C. 3-month time point was minimal, displaying the improved stability of formulations with the addition of acids (data not shown). FIG. 14 shows the data for impurities at 1, 3 and 6 months from the stability study at 25° C., showing that the overall formulation stability can be optimized by adding a specific amount of acid as an excipient. The molar ratio of degarelix API (calculated as free base) to acetic acid additive used in the test formulations shown in Example 2 is between the molar ratios of 1:1 and about 1:3 as shown in FIG. 12, or approximately 1:2. The stability study at 5° C. condition is expected to show a similar pattern of improved stability through the addition of an acid excipient to the formulations.

Overall, this study indicated that an acid excipient or a lowered apparent pH can stabilize the formulation. Without being bound by theory, the ability of an acid to stabilize these formulations could be due to an acid-induced inhibition of peptide hydrolysis and oxidation, two degradant pathways that can be significant sources of degarelix acetate impurity growth.

Various modifications of the above-described invention will be evident to those skilled in the art. It is intended that such modifications are included within the scope of the following claims.

1. A composition, comprising:
a therapeutically effective amount of degarelix or a pharmaceutically acceptable salt thereof; and
an organic biocompatible solvent selected from dimethyl sulfoxide (DMSO) and N-methyl-2-pyrrolidone (NMP),
wherein:
the composition is free of any biodegradable polymer;
the composition is formulated for subcutaneous injection into a subject;
a single dose of the composition is provided in a volume of about 2 mL or less; and
upon a single injection into the subject, the composition forms an in situ depot that releases the degarelix or the pharmaceutically acceptable salt thereof over a time period of at least about 2 months.

2. The composition of claim 1, wherein the in situ depot releases the degarelix over a time period selected from the group consisting of: at least about at least about 2.5 months, at least about 3 months, at least about 3.5 months, at least about 4 months, at least about 4.5 months, and at least about 5 months.

3. The composition of claim 1, wherein the pharmaceutically acceptable salt of degarelix is degarelix acetate.

4-6. (canceled)

7. The composition of claim 1, wherein the therapeutically effective amount of degarelix or the pharmaceutically acceptable salt thereof in the composition is from about 20 wt % to about 40 wt % degarelix free base equivalent.

8-9. (canceled)

10. The composition of claim 1, wherein the therapeutically effective amount of degarelix is from about 120 mg-500 mg.

11-12. (canceled)

13. The composition of claim 1, wherein a single dose of the composition is provided in a volume of about 1.5 mL or less.

14-17. (canceled)

18. The composition of claim 1, wherein the composition further comprises one or more additives selected from an acid additive and an alcohol additive.

19-24. (canceled)

25. The composition of claim 18, wherein the composition comprises from about 0.1 wt % to about 10.0 wt % of the acid additive.

26. The composition of claim 18, wherein the composition comprises from about 1.0 wt % to about 30 wt %, of the alcohol additive.

27-30. (canceled)

31. The composition of claim 18, wherein the acid additive is selected from the group consisting of acetic acid, citric acid, and succinic acid.

32. (canceled)

33. The pharmaceutical composition of claim 18, wherein the alcohol additive is benzyl alcohol (BnOH).

34-40. (canceled)

41. The composition of claim 1, wherein the amount of degarelix or the pharmaceutically acceptable salt thereof is from about 31 wt % to about 34 wt % degarelix free base equivalent, or from about 31 wt % to about 33 wt % degarelix free base equivalent, or from about 31 wt % to about 32 wt % degarelix free base equivalent.

42-45. (canceled)

46. The composition of claim 1, wherein the amount of degarelix or the pharmaceutically acceptable salt thereof is from about 20 wt % to about 23 wt % degarelix free base equivalent, or from about 20 wt % to about 22 wt % degarelix free base equivalent, or from about 21 wt % to about 23 wt % degarelix free base equivalent, or from about 21 wt % to about 22 wt % degarelix free base equivalent.

47. A method of treating prostate cancer in a subject, comprising subcutaneously administering the composition of claim 1 to the subject.

48. The method of claim 47, wherein the prostate cancer is advanced prostate cancer.

49. The method of claim 47, wherein the degarelix or the pharmaceutically acceptable salt thereof in the composition is administered in a dose amount of about 120 mg to about 500 mg.

50. The method of claim 47, wherein the subject's serum testosterone levels are reduced to 50 ng/DL or less.

51. The method of claim 50, wherein the serum testosterone level is reduced to below 20 ng/dL.

52-59. (canceled)

60. The method of claim 47, wherein a single injection of the composition is administered about once every 2 months, about once every 3 months, about once every 4 months, about once every 5 months, or about once every six months.

61. The method of claim 47, wherein a single injection of the composition is administered to the subject about once every three months.

62-71. (canceled)

72. A composition comprising:
(a) degarelix or a pharmaceutically acceptable salt thereof in an amount of from about 31 wt % to about 33 wt % degarelix free base equivalent; and
(b) dimethyl sulfoxide (DMSO);
wherein the composition is free of any biodegradable polymer.

73. The composition of claim 72, wherein the total amount of acid in the composition is from about 1.0 wt % to about 4.0 wt %.

74. The composition of claim 72, wherein the composition comprises about 35 wt % degarelix acetate.

75. A composition comprising:
(a) degarelix of a pharmaceutically acceptable salt thereof in an amount of from about 20 wt % to about 40 wt % degarelix free base equivalent;
(b) N-methyl-2-pyrrolidone (NMP); and
(c) an acid additive or an alcohol additive;
wherein the composition is free of any biodegradable polymer.

76. The composition of claim 75, wherein the amount of degarelix or the pharmaceutically acceptable salt thereof is from about 31 wt % to about 33 wt % degarelix free base equivalent.

77. The composition of claim 75, wherein the composition comprises from about 1.0 wt % to about 5.0 wt % of the acid additive, wherein the acid additive is acetic acid.

78. The composition of claim 75, wherein the total amount of acid in the composition is from about 1.0 wt % to about 10.0 wt %.

79. The composition of claim 75, wherein the composition comprises from about 5.0 wt % to about 30 wt % of the alcohol additive, wherein the alcohol additive is benzyl alcohol (BnOH).
